SEQUENCE LISTING (59 sequences)

SEQ ID NO: 1 - protein, 8 aa, Artificial Sequence
  Synthesized
DTSYGLYS

SEQ ID NO: 2 - protein, 8 aa, Artificial Sequence
  Synthesized
GAKYGLAA

SEQ ID NO: 3 - protein, 8 aa, Artificial Sequence
  Synthesized
GAKYGLFA

SEQ ID NO: 4 - protein, 8 aa, Artificial Sequence
  Synthesized
GANYGLAA

SEQ ID NO: 5 - protein, 8 aa, Artificial Sequence
  Synthesized
GANYGLAS

SEQ ID NO: 6 - protein, 10 aa, Artificial Sequence
  Synthesized
TTDWERMSIG

SEQ ID NO: 7 - protein, 10 aa, Artificial Sequence
  Synthesized
SSNQERISIS

SEQ ID NO: 8 - protein, 10 aa, Artificial Sequence
  Synthesized
SSNKEQISIS

SEQ ID NO: 9 - protein, 5 aa, Artificial Sequence
  Synthesized
NKRAK

SEQ ID NO: 10 - protein, 5 aa, Artificial Sequence
  Synthesized
NKRTM

SEQ ID NO: 11 - protein, 5 aa, Artificial Sequence
  Synthesized
NKGAK

SEQ ID NO: 12 - protein, 5 aa, Artificial Sequence
  Synthesized
NKGTK

SEQ ID NO: 13 - protein, 17 aa, Artificial Sequence
  Synthesized
QSGMAISTGSGHGYNWY

SEQ ID NO: 14 - protein, 17 aa, Artificial Sequence
  Synthesized
QSGMAIDIGSGHGYNWY

SEQ ID NO: 15 - protein, 10 aa, Artificial Sequence
  Synthesized
YPWAMWGQWY

SEQ ID NO: 16 - protein, 14 aa, Artificial Sequence
  Synthesized
VFMPQHWHPAAHWY

SEQ ID NO: 17 - protein, 12 aa, Artificial Sequence
  Synthesized
REARHPWLRQWY

SEQ ID NO: 18 - protein, 14 aa, Artificial Sequence
  Synthesized
YPWGAGAPWLVQWY

SEQ ID NO: 19 - protein, 25 aa, Artificial Sequence
  Synthesized
ASVNQTPRTATKETGESLTINCVLT

SEQ ID NO: 20 - protein, 25 aa, Artificial Sequence
  Synthesized
AKVDQTPRTATKETGESLTINCVLT

SEQ ID NO: 21 - protein, 25 aa, Artificial Sequence
  Synthesized
TRVDQTPRTATKETGESLTINCVVT

SEQ ID NO: 22 - protein, 25 aa, Artificial Sequence
  Synthesized
TRVDQTPRTATKETGESLTINCVLT

SEQ ID NO: 23 - protein, 25 aa, Artificial Sequence
  Synthesized
ASVNQTPRTATKETGESLTINCVVT

SEQ ID NO: 24 - protein, 25 aa, Artificial Sequence
  Synthesized
TRVDQSPSSLSASVGDRVTITCVLT

SEQ ID NO: 25 - protein, 9 aa, Artificial Sequence
  Synthesized
TSWFRKNPG

SEQ ID NO: 26 - protein, 9 aa, Artificial Sequence
  Synthesized
TYWYRKNPG

SEQ ID NO: 27 - protein, 7 aa, Artificial Sequence
  Synthesized
GRYVESV

SEQ ID NO: 28 - protein, 7 aa, Artificial Sequence
  Synthesized
GRYSESV

SEQ ID NO: 29 - protein, 21 aa, Artificial Sequence
  Synthesized
SFSLRIKDLTVADSATYYCKA

SEQ ID NO: 30 - protein, 21 aa, Artificial Sequence
  Synthesized
SFTLTISSLQPEDSATYYCRA

SEQ ID NO: 31 - protein, 21 aa, Artificial Sequence
  Synthesized
SFTLTISSLQPEDFATYYCKA

SEQ ID NO: 32 - protein, 10 aa, Artificial Sequence
  Synthesized
DGAGTVLTVN

SEQ ID NO: 33 - protein, 10 aa, Artificial Sequence
  Synthesized
DGAGTKVEIK

SEQ ID NO: 34 - protein, 15 aa, Artificial Sequence
  Synthesized
YMESLHMQGEIENQI

SEQ ID NO: 35 - protein, 20 aa, Artificial Sequence
  Synthesized
CQPWNSQYPHTHTFTALRFP

SEQ ID NO: 36 - protein, 23 aa, Artificial Sequence
  Synthesized
RSTIYGSRLRIRNLDTTDTGYFQ

SEQ ID NO: 37 - protein, 36 aa, Artificial Sequence
  Synthesized
QCVATNGKEVVSSTGVLFVKFGPPPTASPGYSDEYE

SEQ ID NO: 38 - skipped

SEQ ID NO: 39 - protein, 112 aa, Artificial Sequence
  Synthesized
ASVNQTPRTATKETGESLTINCVLTDTSYGLYSTSWFRKNPGTTDWERMSIGGRYVESVNKRAKSFSLRIKDLTVADSATYYCKAQSGMAISTGSGHGYNWYDGAGTVLTVN

SEQ ID NO: 40 - protein, 112 aa, Artificial Sequence
  Synthesized
AKVDQTPRTATKETGESLTINCVLTDTSYGLYSTSWFRKNPGTTDWERMSIGGRYVESVNKRAKSFSLRIKDLTVADSATYYCKAQSGMAIDIGSGHGYNWYDGAGTVLTVN

SEQ ID NO: 41 - protein, 105 aa, Artificial Sequence
  Synthesized
TRVDQTPRTATKETGESLTINCVVTGAKYGLAATYWYRKNPGSSNQERISISGRYVESVNKRTMSFSLRIKDLTVADSATYYCKAYPWAMWGQWYDGAGTVLTVN

SEQ ID NO: 42 - protein, 109 aa, Artificial Sequence
  Synthesized
TRVDQTPRTATKETGESLTINCVVTGAKYGLFATYWYRKNPGSSNQERISISGRYVESVNKRTMSFSLRIKDLTVADSATYYCKAVFMPQHWHPAAHWYDGAGTVLTVN

SEQ ID NO: 43 - protein, 107 aa, Artificial Sequence
  Synthesized
TRVDQTPRTATKETGESLTINCVLTDTSYGLYSTSWFRKNPGTTDWERMSIGGRYVESVNKGAKSFSLRIKDLTVADSATYYCKAREARHPWLRQWYDGAGTVLTVN

SEQ ID NO: 44 - protein, 109 aa, Artificial Sequence
  Synthesized
ASVNQTPRTATKETGESLTINCVVTGANYGLAATYWYRKNPGSSNQERISISGRYVESVNKRTMSFSLRIKDLTVADSATYYCKAYPWGAGAPWLVQWYDGAGTVLTVN

SEQ ID NO: 45 - protein, 109 aa, Artificial Sequence
  Synthesized
TRVDQSPSSLSASVGDRVTITCVLTGANYGLASTYWYRKNPGSSNKEQISISGRYSESVNKGTKSFTLTISSLQPEDSATYYCRAYPWGAGAPWLVQWYDGAGTKVEIK

SEQ ID NO: 46 - protein, 109 aa, Artificial Sequence
  Synthesized
TRVDQSPSSLSASVGDRVTITCVLTGANYGLASTYWYRKNPGSSNQERISISGRYSESVNKRTMSFTLTISSLQPEDSATYYCRAYPWGAGAPWLVQWYDGAGTKVEIK

SEQ ID NO: 47 - protein, 107 aa, Artificial Sequence
  Synthesized
TRVDQSPSSLSASVGDRVTITCVLTDTSYGLYSTSWFRKNPGTTDWERMSIGGRYVESVNKGAKSFTLTISSLQPEDFATYYCKAREARHPWLRQWYDGAGTKVEIK

SEQ ID NO: 48 - protein, 107 aa, Artificial Sequence
  Synthesized
TRVDQSPSSLSASVGDRVTITCVLTDTSYGLYSTYWYRKNPGSSNKEQISISGRYSESVNKGTKSFTLTISSLQPEDSATYYCRAREARHPWLRQWYDGAGTKVEIK

SEQ ID NO: 49 - protein, 107 aa, Artificial Sequence
  Synthesized
TRVDQSPSSLSASVGDRVTITCVLTDTSYGLYSTYWYRKNPGTTDWERMSIGGRYSESVNKGAKSFTLTISSLQPEDSATYYCRAREARHPWLRQWYDGAGTKVEIK

SEQ ID NO: 50 - protein, 12 aa, Artificial Sequence
  Synthesized
PGVQPSPGGGGS

SEQ ID NO: 51 - protein, 12 aa, Artificial Sequence
  Synthesized
PGVQPAPGGGGS

SEQ ID NO: 52 - protein, 25 aa, Artificial Sequence
  Synthesized
QACKAHHHHHHGAEFEQKLISEEDL

SEQ ID NO: 53 - protein, 25 aa, Artificial Sequence
  Synthesized
QACGAHHHHHHGAEFEQKLISEEDL

SEQ ID NO: 54 - protein, 107 aa, Artificial Sequence
  Synthesized
TRVDQTPRTATKETGESLTINCVLTDTSYGLYSTSWFRKNPGTTDWERMSIGGRYVESVNKGAKSFSLRIKDLTVADSATYYCKAQSLAISTRSYWYDGAGTVLTVN

SEQ ID NO: 55 - protein, 103 aa, Artificial Sequence
  Synthesized
TRVDQSPSSLSASVGDRVTITCVLTDTSYPLYSTYWYRKNPGSSNKEQISISGRYSESVNKGTKSFTLTISSLQPEDSATYYCRAMSTNIWTGDGAGTKVEIK

SEQ ID NO: 56 - protein, 25 aa, artificial sequence
  Synthesized
ASVTQSPRSASKETGESLTITCRVT

SEQ ID NO: 57 - protein, 21 aa, artificial sequence
  Synthesized
SFSLRISSLTVEDSATYYCKA

SEQ ID NO: 58 - protein, 10 aa, artificial sequence
  Synthesized
DGQGTKLEVK

SEQ ID NO: 59 - protein, 109 aa, artificial sequence
  Synthesized
ASVTQSPRSASKETGESLTITCRVTGANYGLAATYWYRKNPGSSNQERISISGRYSESVNKRTMSFSLRISSLTVEDSATYYCKAYPWGAGAPWLVQWYDGQGTKLEVK

(19) United States
(12) Patent Application Publication
(10) Pub. No.: US 2023/0241072 A1
(43) Pub. Date: Aug. 3, 2023

(54) ANTHRACYCLINE DERIVATIVES

(71) Applicant: ALMAC DISCOVERY LIMITED, Craigavon, Northern Ireland (GB)

(72) Inventors: Graham COTTON, Craigavon (GB); Jennifer THOM, Craigavon (GB); Paul TRUMPER, Craigavon (GB)

(21) Appl. No.: 17/620,819

(22) PCT Filed: Jun. 19, 2020
(86) PCT No.: PCT/EP2020/067210

§ 371 Date: Dec. 20, 2021

(30) Foreign Application Priority Data
Jun. 20, 2019 (GB) 1908886.3

(51) Int. Cl. A61K 31/5377; A61K 47/10; A61K 47/68; A61P 35/00

(52) U.S. Cl. CPC A61K 31/5377; A61K 47/10; A61K 47/6849; A61P 35/00

(57) ABSTRACT

The present application relates to anthracycline derivatives and their use in forming conjugates with target-binding molecules, including but not limited to antibodies. Conjugates of target-binding molecules and anthracycline derivatives are also provided. Also provided are medical uses of and pharmaceutical compositions comprising the conjugates.

### RELATED APPLICATIONS

The present application is a U.S. National Stage application under 35 USC 371 of PCT Application Serial No. PCT/EP2020/067210, filed on 19 Jun. 2020; which claims priority from GB Patent Application No. 1908886.3 filed 20 Jun. 2019, the entirety of both of which are incorporated herein by reference.

### SEQUENCE LISTING

The instant application contains a Sequence Listing, which has been submitted electronically in ASCII format and is hereby incorporated by reference in its entirety. Said ASCII copy, created on Mar. 28, 2023, is named 17620819_ST25.txt and is 25,035 bytes in size.

### FIELD OF INVENTION

The present invention relates to anthracycline derivatives and their use in forming conjugates with target-binding molecules, including but not limited to antibodies. Conjugates of target-binding molecules and anthracycline derivatives are also provided.

### INTRODUCTION

Conjugates of small molecular weight toxins to specific binding proteins such as antibodies, are powerful tools to direct toxic payloads to targets within the body. An example is the use of such conjugates to target toxic payloads to cancer cells and such conjugates show great promise in the treatment of cancer.

In order to develop effective and safe conjugates for cancer therapy, several aspects need to be addressed. First, the binding protein or antibody needs to be specific for a given tumour specific antigen (TSA), which should hardly or ideally not be expressed by normal or healthy tissue cells.

Second, the covalent bond, or linkage, between the drug and the binding protein needs to be stable in circulation, preventing undesired release of the toxic payload in the blood stream, but it has to effectively release the drug upon binding to and/or internalization into the cancer cells. Third, the toxic payload has to be of high enough toxicity, or potency, in order to effect the destruction of the cancer cells, even if potentially limited amounts of the TSA are expressed on the cancer cells and therefore only limited amounts of the ADC are internalized, or if release of the toxic payload is not effected at high enough efficiency upon binding to the cancer cells, or upon internalization into the cancer cell.

The anthracycline derivative PNU-159682 has been described as a metabolite of nemorubicin (Quintieri et al. (2005) Clin. Cancer Res. 11, 1608-1617) and has been reported to exhibit extremely high potency for in vitro cell killing in the pico- to femtomolar range with one ovarian (A2780) and one breast cancer (MCF7) cell line (WO2012/073217 A1). Derivatives of PNU-159682 have also been described in WO2016/102679.

Conjugation of PNU-159682 derivatives to antibodies is described in WO2009/099741, WO2016/127081 and WO2016/102679, Yu et al, Clin. Cancer Res 2015, 21, 3298 and Stefan et al, Mol. Cancer. Ther., 2017, 16,879.

### SUMMARY OF INVENTION

The present invention provides anthracycline (PNU) derivatives suitable for use in drug conjugates. Specifically, derivatives of PNU159682 are provided, which lack the C14 carbon and attached hydroxyl functionality, and in which an ethylenediamino (EDA) group forms part of a linker region between the C13 carbonyl of PNU159682 and a maleimide group. Where the linker comprises val-cit-PAB the maleimide group may be replaced with any reactive group suitable for a conjugation reaction. Such payloads are able to react with a free thiol group on another molecule. Where the free thiol is on a protein a protein-drug conjugate (PDC) may be formed.

Accordingly, in a first aspect there is provided an anthracycline (PNU) derivative of formula (I):

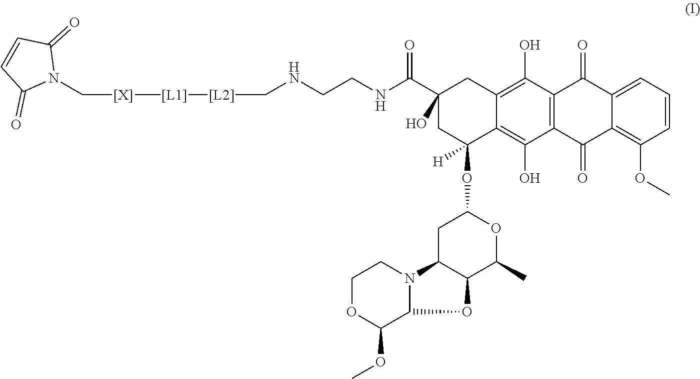

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [12] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, any amino acid except glycine, and combinations thereof.

The anthracycline (PNU) derivative of formula (I) may comprise [L1], [12] or [L1] and [12].

Preferably, where [L1] and/or [12] are peptides, said peptides do not contain glycine.

It will be clear to those of skill in the art that when optional spacers and/or optional linkers are absent a bond remains in their place.

Preferably, [X] is selected from the group comprising polyethylene glycol,

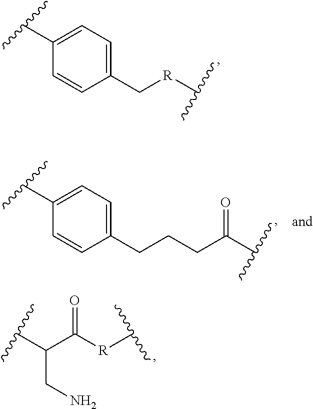

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

Preferably, [12] is p-aminobenzyloxycarbonyl (PAB) or Alanine.

Preferably, the PNU derivative has a structure selected from:

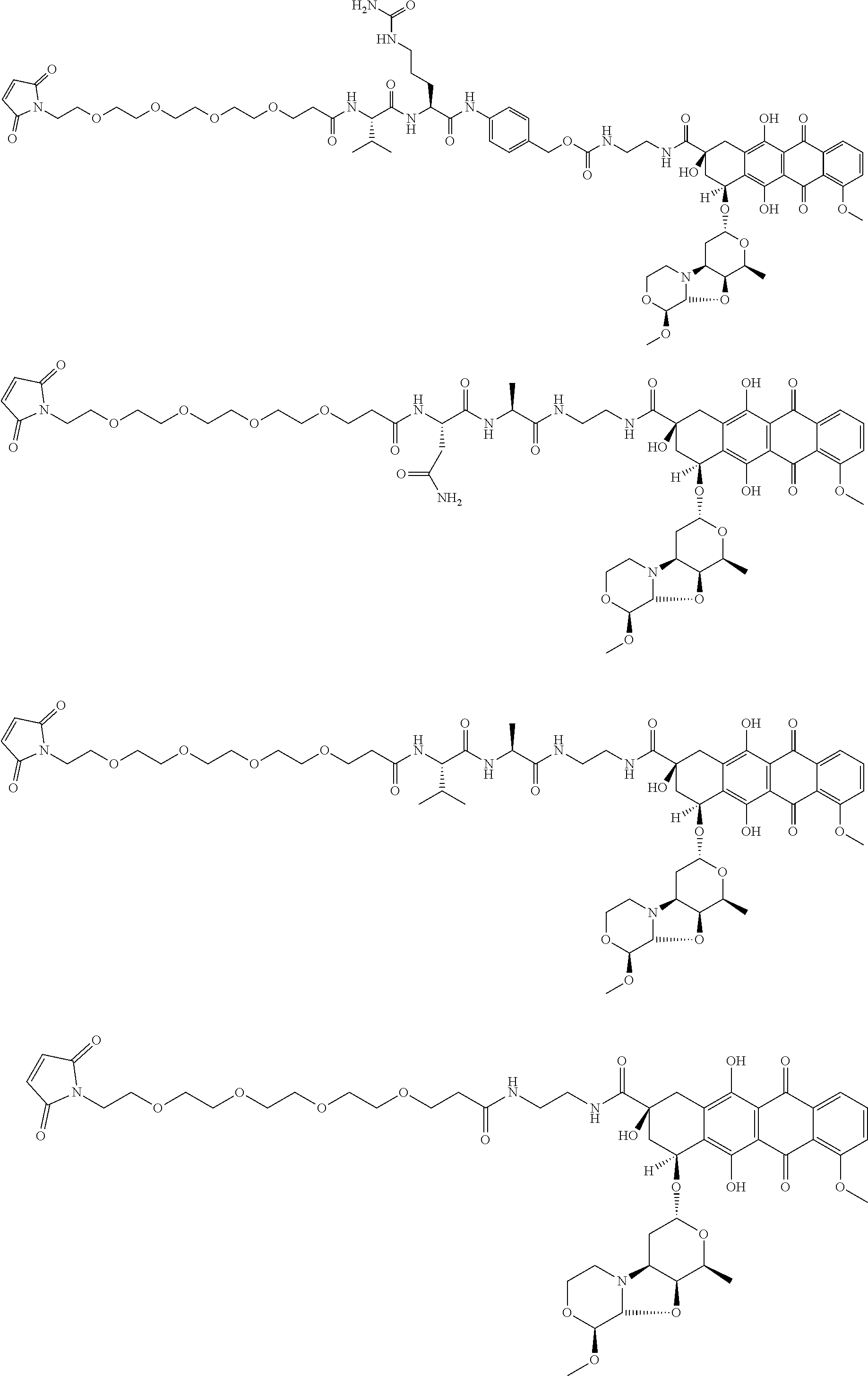

In a second aspect there is provided an anthracycline (PNU) derivative of formula (IV):

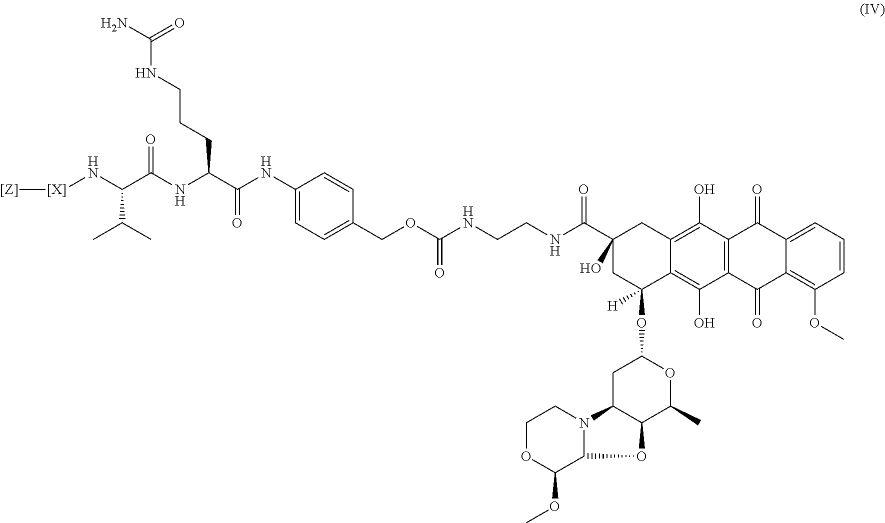

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
wherein [Z] is a reactive group. The reactive group may be any reactive group suitable for use in a conjugation reaction, particularly a conjugation reaction to a target binding molecule.
[Z] may therefore be a moiety comprising a functional group for use in bioconjugation reactions. Functional groups for use in bioconjugation reactions include but are not limited to,
maleimides or alkyl halides for reaction with thiol groups or selenol groups on proteins through thioether and selonoether reactions;
sulphydryl groups for reaction with maleimide, alkyl halide or thiol functionalised molecules including the thiol groups of protein cysteine residues;
activated disulphides such as pyridyl dithiols (Npys thiols) or TNB thiols (5-thiol-2-nitrobenzoic acid) for reaction with thiol groups to form disulphide linkages through thiol disulphide exchange;
amino groups for attachment to carboxyl groups on proteins and biomolecules through amide bond forming reactions;
alkyne groups, particularly ring constrained alkynes such as dibenzocyclooctyne (DBCO) or bicyclo[6.1.0]nonyne (BCN) for the reaction with azido functionalised biomolecules through strain promoted alkyne-azide cycloaddition copper free chemistry. Azido functionalities can be introduced into proteins through, for example, the incorporation of the unnatural amino acid para-azidomethy-L-phenyalanine or into protein glycans using enzyme mediated glycoengineering to attach azido-containing sugar analogues;
azido groups for reaction with alkyne functionalised target-binding molecule through strain promoted alkyne-azide cycloaddition copper free chemistry;
aminoxy groups for reactions with aldehyde and ketone groups on biomolecules through oxime forming ligations. Ketones can be introduced into proteins through the use of amber stop codon technologies such as the incorporation of the non-natural amino acid, para-acetyl phenylalanine. Aldehydes can be found on biomolecules through the presence of reducing sugars and can be introduced into proteins through periodate oxidation of N-terminal serine residues or periodate oxidation of cis-glycol groups of carbohydrates. Aldehyde groups can also be incorporated into proteins through the conversion of protein cysteines, within specific sequences, to formyl glycine by formylglycine generating enzyme. In addition formylglycine containing proteins have been conjugation to payloads via the Hydrazino-Pictet-Spengler (HIPS) ligation;
aldehyde or ketone groups for the reaction with aminoxy or hydrazide or hydrazinyl functionalized biomolecules through oxime or hydrazine bond forming ligation reactions. Protein aminoxy and hydrazide functionalized proteins can be generated through cleavage of intein-fusion proteins.
   [Z] may therefore be selected from the group consisting of a maleimide, an alkyl halide, a sulphydryl group, an activated disulphide (such as pyridyl dithiols (Npys thiols) or TNB thiols (5-thiol-2-nitrobenzoic acid)), an amino group, an alkyne group (such as ring constrained alkynes such as dibenzocyclooctyne (DBCO) or bicyclo[6.1.0]nonyne (BCN)), an azido group, an aminoxy group, an aldehyde group and a ketone group.
   [Z] may also be a moiety for enzyme mediated bioconjugation reactions. Moieties for use in enzyme mediated conjugation reactions include but are not limited to polyGly [(Gly)ₙ] for use in sortase-enzyme mediated antibody conjugation or an appropriate primary amine for bacterial transglutaminase mediated conjugation to glutamine γ-carboxyamide groups contained with sequences such as Lys-Lys-Gin-Gly and Lys-Pro-Glu-Thr-Gly.
   [Z] may therefore be selected from the group consisting of polyGly and a primary amine.

The PNU derivative according to the second aspect of the invention may therefore correspond to a PNU derivative of the first aspect of the invention wherein L1 is Val-Cit-PAB, L2 is absent and wherein the maleimide group may be replaced with another Reactive Group as defined above.

Preferably, [X] is selected from the group comprising polyethylene glycol,

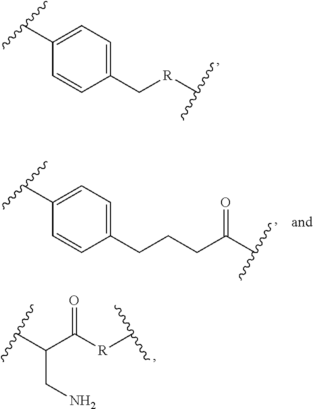

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

The PNU derivative of the first aspect may be conjugated to a variety of moieties. In particular, the PNU derivative of the first aspect may be conjugated to a molecule that binds a target, referred to herein as a target-binding molecule. The PNU derivative of the second aspect may be conjugated to a variety of moieties. In particular, the PNU derivative of the second aspect may be conjugated to a molecule that binds a target, referred to herein as a target-binding molecule. Examples of target-binding molecules include, but are not limited to biomolecules, peptides, small molecules, proteins, and nucleic acids (including but not limited to aptamers). In some cases, the target-binding molecule may be multimeric (for example dimers, trimers and higher-order multimers or multi-subunit proteins).

Accordingly, in a further aspect there is provided a target-binding molecule-drug conjugate comprising the PNU according to the first aspect and a binding molecule. Alternatively, in this aspect there is provided a target-binding molecule-drug conjugate comprising the PNU according to the second aspect and a binding molecule. Binding molecules suitable for use in this aspect include but are not limited to biomolecules, peptides, small molecules, proteins, nucleic acids (including but not limited to aptamers). In accordance with this aspect there is provided a target-binding molecule-drug conjugate, comprising a target-binding molecule and an anthracycline (PNU) derivative, wherein the target-binding molecule-drug conjugate has the structure of formula (II):

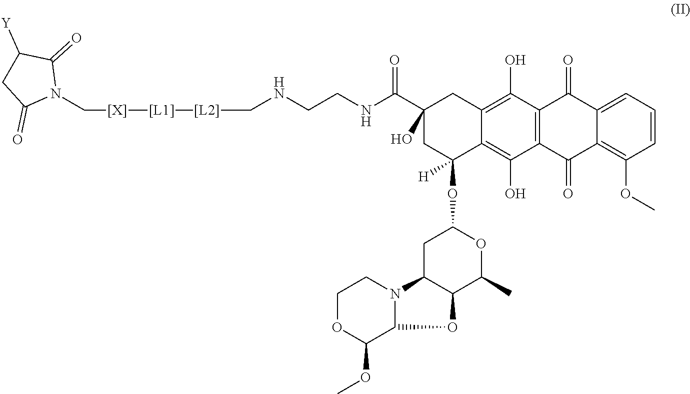

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, any amino acid except glycine, and combinations thereof; and
Y is a target binding molecule.

The target-binding molecule-drug conjugate of formula (II) may comprise [L1], [L2] or [L1] and [L2].

Preferably, target-binding molecule-drug conjugate where [L1] and/or [L2] are peptides, said peptides do not contain glycine.

It will be clear to those of skill in the art that when optional spacers and/or optional linkers are absent a bond remains in their place.

In one embodiment, the anthracycline (PNU) derivative comprises [L1] and/or [12] and [X] is optional. Accordingly, [L1] and/or [12] may be linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, any amino acid except glycine, and combinations thereof. The anthracycline (PNU) derivative of formula (I) may comprise [L1], [12] or [L1] and [12]. The anthracycline (PNU) derivative of formula (I) may comprise [L1] and/or [L2].

In one embodiment, there is provided an anthracycline (PNU) derivative of formula (I):

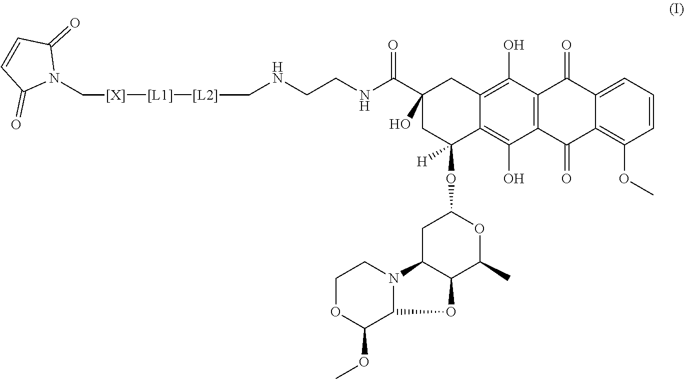

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and/or [12] are linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, any amino acid except glycine, and combinations thereof;
wherein the anthracycline (PNU) derivative of formula (I) comprises [L1], [L2] or [L1] and [L2].

Preferably, [X] is selected from the group comprising polyethylene glycol,

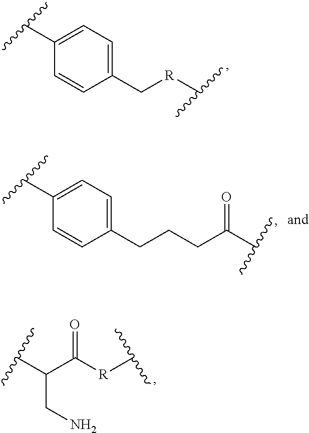

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

Preferably, [12] is p-aminobenyloxycarbonyl (PAB) or Alanine.

Preferably, the PNU derivative has a structure selected from:

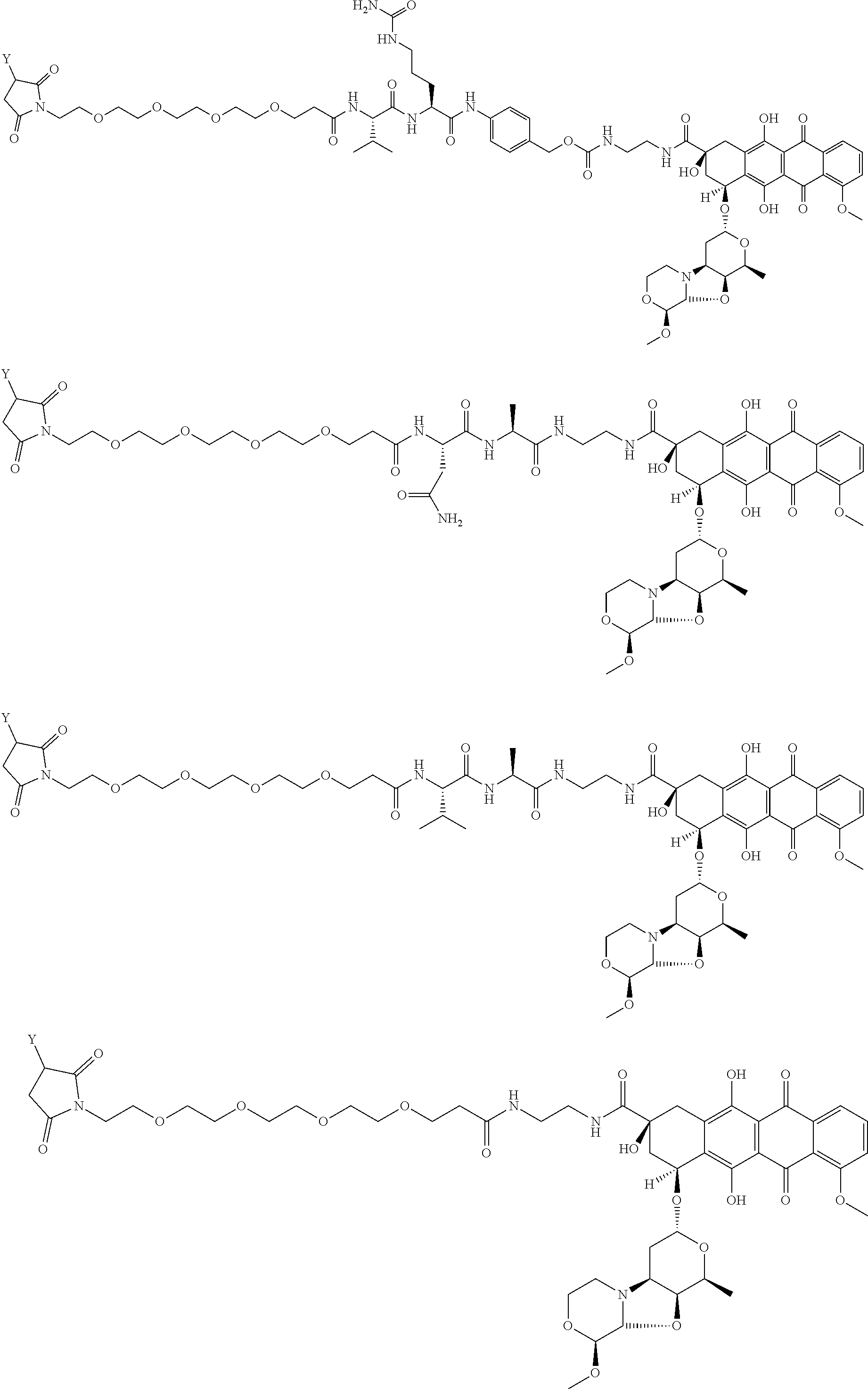

In accordance with this aspect there is also provided a target-binding molecule-drug conjugate, comprising a target-binding molecule and an anthracycline (PNU) derivative, wherein the target-binding molecule-drug conjugate has the structure of formula (V):

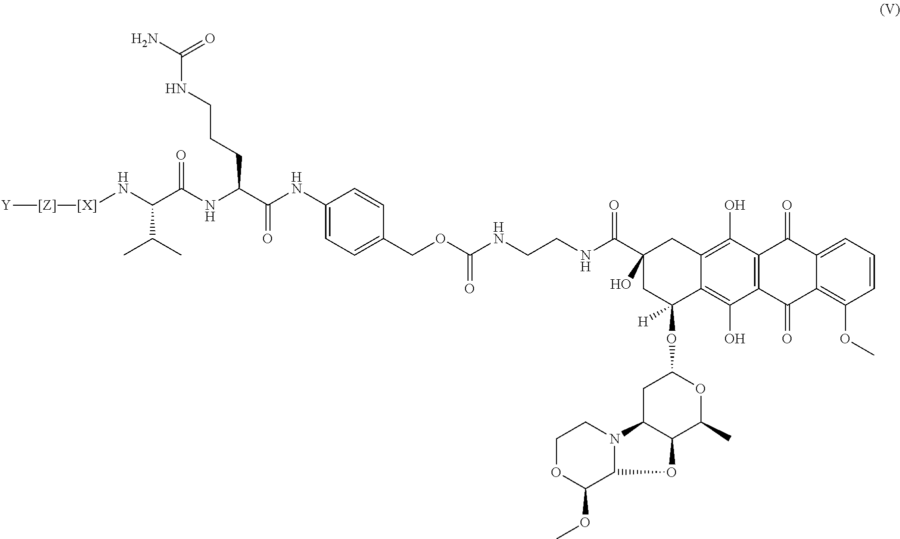

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[Z] is a linker derived from a reactive group used to conjugate the anthracycline (PNU) derivative and the target-binding molecule; and
Y is the target binding molecule.
[Z] is a typically a moiety derived from a reactive group used to conjugate the anthracycline (PNU) derivative and the target-binding molecule. [Z] may be a moiety derived from a reactive group selected from the group consisting of a maleimide, an alkyl halide, a sulphydryl group, an activated disulphide, an amino group, an alkyne group, an azido group, an aminoxy group, an aldehyde group and a ketone group.
[Z] may therefore be selected from the group consisting of a disulphide bond, an amide bond, an oxime bond, a hydrazone bond, a thioether bond, a 1, 2, 3 triazole and polyGly.

Preferably, [X] is selected from the group comprising polyethylene glycol,

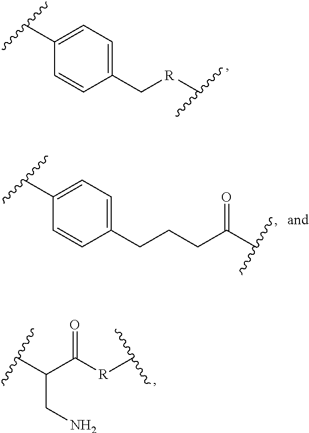

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

Preferably, the target-binding molecule is a protein or a nucleic acid. Examples of target-binding proteins (which may also be referred to as specific antigen binding proteins) include but are not limited to an immunoglobulin or antibody, an immunoglobulin Fc region, an immunoglobulin Fab region, a Fab′, a Fv, a Fv-Fc, a single chain Fv (scFv), a scFv-Fc, (scFv)₂, a diabody, a triabody, a tetrabody, a bispecific t-cell engager (BiTE), an intein, a VNAR domain, a single domain antibody (sdAb), a VH domain, a scaffold protein (affibodies, centyrins, darpins etc.). Examples of target-binding nucleic acids include but are not limited to aptamers.

Preferably, the target-binding molecule-drug conjugate is a protein and the anthracycline (PNU) derivative is conjugated to a thiol-containing amino acid residue in the amino acid sequence of a protein or to a thiol group introduced by chemical modification of the protein, for example incorporated at the N-terminus or C-terminus of the amino acid sequence of the specific antigen binding protein. Thiol groups may also be introduced into other target-binding molecules, such as nucleic acids.

Target-binding proteins (also termed specific antigen binding proteins) may be selected from the group comprising an immunoglobulin or antibody, an immunoglobulin Fc region, an immunoglobulin Fab region, a Fab′, a Fv, a Fv-Fc, a single chain Fv (scFv), a scFv-Fc, (scFv)₂, a diabody, a triabody, a tetrabody, a bispecific t-cell engager (BiTE), an intein, a VNAR domain, a single domain antibody (sdAb), a VH domain, or a scaffold protein (affibodies, centyrins, darpins etc.).

In a preferred embodiment, the target-binding molecule may comprise a specific antigen binding protein may comprise an amino acid sequence represented by the formula (III):

FW1-CDR1-FW2-HV2-FW3a-HV4-FW3b-CDR3-FW4 (III)

wherein
FW1 is a framework region
CDR1 is a CDR sequence
FW2 is a framework region
HV2 is a hypervariable sequence
FW3a is a framework region
HV4 is a hypervariable sequence
FW3b is a framework region
CDR3 is a CDR sequence
FW4 is a framework region.

Preferably, the specific antigen binding protein binds receptor tyrosine kinase-like orphan receptor 1 (ROR1). Preferably, the ROR1-specific antigen binding molecule does not bind to receptor tyrosine kinase-like orphan receptor 2 (ROR2). Preferably the ROR1-specific antigen binding molecule binds to both human ROR1 and murine ROR1 (mROR1). Preferably the ROR1-specific antigen binding molecule binds to deglycosylated ROR1. Such molecules are described in co-pending International patent application no. PCT/EP2018/086823, the content of which is incorporated herein by reference.

More preferably, the ROR1-specific antigen binding molecule does not bind to a linear peptide sequence selected from:

(SEQ ID NO: 34)
YMESLHMQGEIENQI
(SEQ ID NO: 35)
CQPWNSQYPHTHTFTALRFP
(SEQ ID NO: 36)
RSTIYGSRLRIRNLDTTDTGYFQ
(SEQ ID NO: 37)
QCVATNGKEVVSSTGVLFVKFGPPPTASPGYSDEYE

In this embodiment of the target-binding molecule-drug conjugate, the specific antigen binding protein may comprise:
FW1 is a framework region of from 20 to 28 amino acids
CDR1 is a CDR sequence selected from DTSYGLYS (SEQ ID NO: 1), GAKYGLAA (SEQ ID NO: 2), GAKYGLFA (SEQ ID NO: 3), GANYGLAA (SEQ ID NO: 4), or GANYGLAS (SEQ ID NO: 5)
FW2 is a framework region of from 6 to 14 amino acids
HV2 is a hypervariable sequence selected from TTDWERMSIG (SEQ ID NO: 6), SSNQERISIS (SEQ ID NO: 7), or SSNKEQISIS (SEQ ID NO: 8)
FW3a is a framework region of from 6 to 10 amino acids
HV4 is a hypervariable sequence selected from NKRAK (SEQ ID NO: 9), NKRTM (SEQ ID NO: 10), NKGAK (SEQ ID NO: 11), or NKGTK (SEQ ID NO: 12)
FW3b is a framework region of from 17 to 24 amino acids

CDR3 is a CDR sequence selected from
(SEQ ID NO: 13)
QSGMAISTGSGHGYNWY,
(SEQ ID NO: 14)
QSGMAIDIGSGHGYNWY,
(SEQ ID NO: 15)
YPWAMWGQWY,
(SEQ ID NO: 16)
VFMPQHWHPAAHWY,
(SEQ ID NO: 17)
REARHPWLRQWY,
or
(SEQ ID NO: 18)
YPWGAGAPWLVQWY

FW4 is a framework region of from 7 to 14 amino acids
   or a functional variant thereof with at least 45% sequence identity thereto,

More preferably FW1 is selected from: ASVNQTPRTATKETGESLTINCVLT (SEQ ID NO: 19), AKVDQTPRTATKETGESLTINCVLT (SEQ ID NO: 20), TRVDQTPRTATKETGESLTINCVVT (SEQ ID NO: 21), TRVDQTPRTATKETGESLTINCVLT (SEQ ID NO: 22), ASVNQTPRTATKETGESLTINCVVT (SEQ ID NO: 23), TRVDQSPSSLSASVGDRVTITCVLT (SEQ ID NO: 24) or ASVTQSPRSASKETGESLTITCRVT (SEQ ID NO: 56), FW2 is selected from: TSWFRKNPG (SEQ ID NO: 25), or TYWYRKNPG (SEQ ID NO: 26); FW3a is selected from: GRYVESV (SEQ ID NO: 27), or GRYSESV (SEQ ID NO: 28), FW3b is selected from: SFSLRIKDLTVADSATYYCKA (SEQ ID NO: 29), SFTLTISSLQPEDATYYCRA (SEQ ID NO: 30), SFTLTISSLQPEDFATYYCKA (SEQ ID NO: 31) or SFSLRISSLTVEDSATYYCKA (SEQ ID NO: 57), and FW4 is selected from: DGAGTVLTVN (SEQ ID NO: 32), DGAGTKVEIK (SEQ ID NO: 33) or DGQGTKLEVK (SEQ ID NO: 58); or functional variants thereof with a sequence identity of at least 45%.

More preferably the ROR1-specific antigen binding molecule comprises an amino acid sequence selected from:

(SEQ ID NO: 39)
ASVNQTPRTATKETGESLTINCVLTDTSYGLYSTSWFRKNPGTTDWERM
SIGGRYVESVNKRAKSFSLRIKDLTVADSATYYCKAQSGMAISTGSGHG
YNWYDGAGTVLTVN;
(SEQ ID NO: 40)
AKVDQTPRTATKETGESLTINCVLTDTSYGLYSTSWFRKNPGTTDWERM
SIGGRYVESVNKRAKSFSLRIKDLTVADSATYYCKAQSGMAIDIGSGHG
YNWYDGAGTVLTVN;
(SEQ ID NO: 41)
TRVDQTPRTATKETGESLTINCVVTGAKYGLAATYWYRKNPGSSNQERI
SISGRYVESVNKRTMSFSLRIKDLTVADSATYYCKAYPWAMWGQWYDGA
GTVLTVN;
(SEQ ID NO: 42)
TRVDQTPRTATKETGESLTINCVVTGAKYGLFATYWYRKNPGSSNQERI
SISGRYVESVNKRTMSFSLRIKDLTVADSATYYCKAVFMPQHWHPAAHW
YDGAGTVLTVN;
(SEQ ID NO: 43)
TRVDQTPRTATKETGESLTINCVLTDTSYGLYSTSWFRKNPGTTDWERM
SIGGRYVESVNKGAKSFSLRIKDLTVADSATYYCKAREARHPWLRQWYD
GAGTVLTVN;
(SEQ ID NO: 44)
ASVNQTPRTATKETGESLTINCVVTGANYGLAATYWYRKNPGSSNQERI
SISGRYVESVNKRTMSFSLRIKDLTVADSATYYCKAYPWGAGAPWLVQW
YDGAGTVLTVN;,
(SEQ ID NO: 45)
TRVDQSPSSLSASVGDRVTITCVLTGANYGLASTYWYRKNPGSSNKEQI
SISGRYSESVNKGTKSFTLTISSLQPEDSATYYCRAYPWGAGAPWLVQW
YDGAGTKVEIK;
(SEQ ID NO: 46)
TRVDQSPSSLSASVGDRVTITCVLTGANYGLASTYWYRKNPGSSNQERI
SISGRYSESVNKRTMSFTLTISSLQPEDSATYYCRAYPWGAGAPWLVQW
YDGAGTKVEIK;
(SEQ ID NO: 47)
TRVDQSPSSLSASVGDRVTITCVLTDTSYGLYSTSWFRKNPGTTDWERM
SIGGRYVESVNKGAKSFTLTISSLQPEDFATYYCKAREARHPWLRQWYD
GAGTKVEIK;
(SEQ ID NO: 48)
TRVDQSPSSLSASVGDRVTITCVLTDTSYGLYSTYWYRKNPGSSNKEQI
SISGRYSESVNKGTKSFTLTISSLQPEDSATYYCRAREARHPWLRQWYD
GAGTKVEIK;
(SEQ ID NO: 49)
TRVDQSPSSLSASVGDRVTITCVLTDTSYGLYSTYWYRKNPGTTDWERM
SIGGRYSESVNKGAKSFTLTISSLQPEDSATYYCRAREARHPWLRQWYD
GAGTKVEIK;
(SEQ ID NO: 59)
ASVTQSPRSASKETGESLTITCRVTGANYGLAATYWYRKNPGSSNQERI
SISGRYSESVNKRTMSFSLRISSLTVEDSATYYCKAYPWGAGAPWLVQW
YDGQGTKLEVK;

or a functional variant thereof with a sequence identity of at least 45%.

The ROR1-specific antigen binding molecule may be humanized. The ROR1-specific antigen binding molecule may be de-immunized.

The ROR1-specific antigen binding molecule may also be part of a fusion protein. A preferred fusion protein is an ROR1-specific antigen binding molecule fused to an immunoglobulin Fc region. Preferably the immunoglobulin Fc region is a human immunoglobulin Fc region. In some cases the ROR1-specific antigen binding molecule may be a dimer, trimer or higher order multimer. Such multimers may also be fusion proteins with other molecules, including but not limited to immunoglobulin Fc. The individual domains of a fusion protein may be connected by optional linkers. Linkers may include, but are not limited to (G₄S)₅, PGVQPSPGGGGS (referred to as WbG4S) (SEQ ID NO: 50), and PGVQPAPGGGGS (referred to as WbG4SGM) (SEQ ID NO: 51).

Also provided herein is the target-binding molecule-drug conjugate according to the above aspects, for use in therapy.

Also provided herein is the target-binding molecule-drug conjugate according to the above aspects, for use in the treatment of cancer.

Also provided herein is the use of a target-binding molecule-drug conjugate according to the above aspects in the manufacture of a medicament for the treatment of a disease in a patient in need thereof.

Also provided herein is a method of treatment of a disease in a patient in need of treatment comprising administration to said patient of a therapeutically effective dosage of a target-binding molecule-drug conjugate according to the above aspects. The disease may be cancer.

Preferably, the cancer is a ROR1-positive cancer type. More preferably, the cancer is selected from the group comprising blood cancers such as lymphomas and leukemias, chronic lymphocytic leukaemia (CLL), mantle cell lymphoma (MCL), B-cell acute lymphoblastic leukaemia (B-ALL), marginal zone lymphoma (MZL), non-Hodgkin lymphomas (NHL), acute myeloid leukemia (AML) and solid tumours including neuroblastoma, renal cancer, lung cancer, colon cancer, ovarian cancer, pancreatic cancer, breast cancer, skin cancer, uterine cancer, prostate cancer, thyroid cancer, Head and Neck cancer, bladder cancer, stomach cancer or liver cancer. The cancer may be mesothelioma or triple negative breast cancer (TNBC). The mesothelioma may be pleural mesothelioma.

In an embodiment of the above aspect the target-binding molecule is an antibody. In another embodiment of the above aspect the target-binding molecule binds HER-2. Preferably, the target-binding molecule an antibody that binds HER-2. More preferably, antibody is trastuzumab or a derivative thereof.

Also provided herein is a pharmaceutical composition comprising a target-binding molecule-drug conjugate according to any of the above aspects, and at least one other pharmaceutically acceptable ingredient.

### DESCRIPTION OF FIGURES

FIG. 1—Examples of Payloads released from Val-Cit (vc) PAB PNU conjugates.

FIG. 2—PNU derivatives of the present invention.

FIG. 3—Potency of the B1hFc-na-EDA-PNU conjugate in killing ROR1 positive PA-1 cell line versus non-binding control (2VhFc-na-EDA-PNU)

FIG. 4—Potency of the B1hFc-va-EDA-PNU conjugate in killing ROR1 positive PA-1 cell line versus non-binding control (2VhFc-va-EDA-PNU)

FIG. 5—Potency of the B1hFc-na-EDA-PNU conjugate in killing PA-1 cell line with ROR1 knock out versus non-binding control (2VhFc-va-EDA-PNU). The B1hFc-na-EDA-PNU conjugate in PA-1 cell line with ROR1 knock out shows no cell killing.

FIG. 6—Potency of the B1hFc-va-EDA-PNU conjugate in killing PA-1 cell line with ROR1 knock out versus non-binding control (2VhFc-va-EDA-PNU). The B1hFc-va-EDA-PNU conjugate in PA-1 cell line with ROR1 knock out shows no cell killing.

FIG. 7—Potency of the B1hFc-na-EDA-PNU conjugate in killing Kasumi-2 cell line versus non-binding control (2VhFc-na-EDA-PNU)

FIG. 8—Potency of the B1hFc-va-EDA-PNU conjugate in killing Kasumi-2 cell line versus non-binding control (2VhFc-va-EDA-PNU)

FIG. 9—Potency of the B1hFc-na-EDA-PNU conjugate in killing MHH-ES1 cell line versus non-binding control (2VhFc-na-EDA-PNU)

FIG. 10—Potency of the B1hFc-va-EDA-PNU conjugate in killing MHH-ES1 cell line versus non-binding control (2VhFc-va-EDA-PNU)

FIG. 11—Potency of the B1hFc-vc-PAB-EDA-PNU conjugate in killing ROR1 positive PA-1 cell line versus non-binding control (2VhFc-vc-PAB-EDA-PNU)

FIG. 12—Potency of the B1hFc-vc-PAB-EDA-PNU conjugate in PA-1 cell line with ROR1 knock out versus non-binding control (2VhFc-va-EDA-PNU). The B1hFc-vc-PAB-EDA-PNU conjugate in PA-1 cell line with ROR1 knock out shows no cell killing.

FIG. 13—Cell kill data for tras(S442C)-vc-PAB-EDA-PNU conjugate with the Her2 positive cell line SK-BR-3 and Her2 negative cell line MDA-MB-468.

FIG. 14—Cell kill data for tras(S442C)-va-EDA-PNU conjugate with the Her2 positive cell line SK-BR-3 and Her2 negative cell line MDA-MB-468

FIG. 15—Potency of the P3A1hFc(S442C)-va-EDA-PNU conjugate in killing ROR1 positive PA-1 cell-line and PA-1 cell line with ROR1 knock out

FIG. 16—Binding of multimer VNAR conjugates to cell-surface ROR1 (A549 cancer cell-line). (A) BA11-B1-D3 vs BA11-B1-D3-PNU conjugates; (B) P3A1-BA11-D3 vs P3A1-BA11-D3-PNU conjugates; (C) P3A1-BA1-P3A1 vs P3A1-BA11-P3A1-PNU conjugates. Binding to ROR1 is maintained after conjugation with vc-PAB-EDA-PNU or with va-EDA-PNU linker payloads.

FIG. 17—In vivo efficacy of B1-hFc-vc-PAB-EDA-PNU and B1-hFc-va-EDA-PNU evaluated in a PDX model of pleural mesothelioma against vehicle-treated mice. Absolute mean tumour volume plotted+/−standard error of the mean (n=5). Treatment started at a mean tumour volume of 124 mm³. Treatment with PDC molecules was given by iv injection on days 1, 4, 7, 10, 18 (represented by arrows); all mice were pre-primed with mIgG 20h before first PDC dose. Vehicle data plotted to the last day when all mice were alive and present in the group.

FIG. 18—In vivo efficacy of B1-hFc-vc-PAB-EDA-PNU and B1-hFc-va-EDA-PNU evaluated in a PDX model of TNBC against vehicle-treated mice. Absolute mean tumour volume plotted+/−standard error of the mean (n=5). Treatment started at a mean tumour volume of 180 mm³. Treatment with PDC molecules was given by iv injection on days 2, 5, 8, 12, 15 (represented by arrows); all mice were pre-primed with mIgG 20h before first PDC dose. Vehicle data plotted to the last day when all mice were alive and present in the group.

### DETAILED DESCRIPTION

A highly interesting class of DNA intercalating toxins for use as payloads for drug conjugates are anthracyclines, because of their proven clinical validation as chemotherapeutic drugs in cancer therapy. The anthracycline derivative PNU-159682 has been described as a metabolite of nemorubicin (Quintieri et al. (2005) Clin. Cancer Res. 11, 1608-1617) and has been reported to exhibit extremely high potency for in vitro cell killing in the pico- to femtomolar range with one ovarian (A2780) and one breast cancer (MCF7) cell line (WO2012/073217 A1).

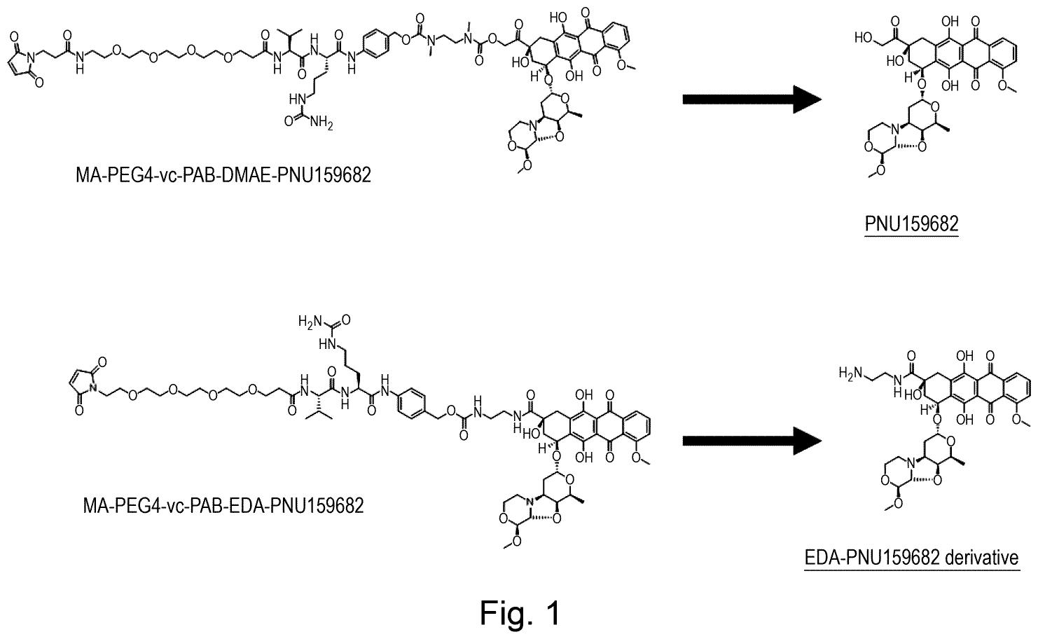

Stability of chemically-conjugated protein drug conjugates is an important consideration, since unintended release of a highly potent anthracycline toxin, like PNU-159682, in the circulation of a patient prior to targeting of the tumour cells would lead to off target effects and undesirable side effects. Some example molecules released from PNU conjugates are given in FIG. 1, which shows release of PNU159682 and an EDA-PNU159682 derivative from different Val-Cit-PAB containing drug linkers.

Potent toxins that can be linked to targeting proteins with high stability are therefore required in order to avoid, or at least reduce, unwanted side effects. Alternatively, linker payloads are designed such that extracellular cleavage releases derivatives of the payload with attenuated potency. However, sufficient potency needs to be retained in order to avoid any reduction in side effect being negated due to the need to administer higher doses to achieve efficacy.

Ease of conjugation is an important factor in producing easily manufacturable products. The payloads of the first aspect of the present invention use a maleimide group, which can react to any available thiol group on a conjugation partner using straightforward and standard conditions. Furthermore, the use of maleimide/thiol chemistry for conjugation allows for site-specific conjugation to introduced thiol groups, for example on the side-chain of an engineered cysteine residue in a protein sequence. In some cases described herein, a cysteine may be introduced via the introduction of his myc tag containing an engineered cysteine (example sequences include, but are not limited to, QACKAHHHHHHGAEFEQKLISEEDL (SEQ ID NO: 52) or QACGAHHHHHHGAEFEQKLISEEDL (SEQ ID NO: 53)) at the C- or N-terminal of a protein.

Antibody/protein drug conjugates generated using non-selective labelling methods, such as through reaction with amino functionalities within proteins, deliver products containing multiple different species with differing drug to antibody ratios. This impacts the properties of the conjugate including potency and PK properties which impacts in vivo efficacy and toxicities. Therefore, thiol reactive payloads are of great importance, as these can be reacted in high yield, in a simple process, with naturally occurring cysteine residues in proteins or with a cysteine residue engineered into a specific site at any point within the sequence of proteins using molecular biology/recombinant protein expression or chemical synthesis or through chemical modification of expressed, synthetic or natural proteins.

The present invention provides anthracycline (PNU) derivatives suitable for use in drug conjugates, including but not limited to protein-drug conjugates (PDCs). Specifically, derivatives of PNU159682 are provided, which lack the C14 carbon and attached hydroxyl functionality, and in which an ethylenediamino (EDA) group forms part of a linker region between the C13 carbonyl of PNU159682 and a maleimide group. A maleimide group is present in the anthracycline (PNU) derivatives of the first aspect of the invention and may also be present in the anthracycline (PNU) derivatives of the second aspect of the invention. Such payloads are able to react with a free thiol group on another molecule. Where the free thiol is on a protein, a protein-drug conjugate (PDC) may be formed.

Surprisingly, derivatives of PNU159682 functionalised with an ethylenediamino (EDA) group and linked to a thiol group via a maleimide group show higher stability compared to non-EDA payloads or liberated payload derivatives with slightly less potency. More stable payloads may be advantageous because of reduced off-target effects, which in turn may lead to reduced side effects and increased patient compliance.

The invention further provides a target-binding molecule—drug conjugate, comprising an anthracycline derivative conjugate according to the above disclosure and a target-binding molecule.

According to another embodiment of the target-binding molecule-drug conjugate, the target-binding molecule is a protein and the anthracycline (PNU) derivative is conjugated, optionally by means of one or more linkers, to a thiol group introduced into the amino acid sequence of the protein. The introduced thiol may be introduced at the amino or carboxy terminus of the protein, or to the amino or carboxy terminus of a domain or subunit thereof. In another embodiment the conjugation is to a thiol group in a sequence introduced at the at the amino or carboxy terminus of the protein, or to the amino or carboxy terminus of a domain or subunit thereof.

The target-binding molecule may be a protein such as a specific antigen binding protein may be a VNAR domain derived from the Novel or New antigen receptor (IgNAR) found in the sera of cartilaginous fish (Greenberg A. S., et al., Nature, 1995. 374(6518): p. 168-173, Dooley, H., et al, Mol. Immunol, 2003. 40(1): p. 25-33; Müller, M. R., et al., mAbs, 2012. 4(6): p. 673-685)).

The specific antigen binding protein may therefore comprise an amino acid sequence represented by the formula (III):

FW1-CDR1-FW2-HV2-FW3a-HV4-FW3b-CDR3-FW4 (III)

wherein
FW1 is a framework region
CDR1 is a CDR sequence
FW2 is a framework region
HV2 is a hypervariable sequence
FW3a is a framework region
HV4 is a hypervariable sequence
FW3b is a framework region
CDR3 is a CDR sequence
FW4 is a framework region.

Framework region FW1 is preferably from 20 to 28 amino acids in length, more preferably from 22 to 26 amino acids in length, still more preferably from 23 to 25 amino acids in length. In certain preferred embodiments, FW1 is 26 amino acids in length. In other preferred embodiments, FW1 is amino acids in length. In still other preferred embodiments, FW1 is 24 amino acids in length.

CDR region CDR1 is preferably from 7 to 11 amino acids in length, more preferably from 8 to 10 amino acids in length. In certain preferred embodiments, CDR1 is 9 amino acids in length. In other preferred embodiments, CDR1 is 8 amino acids in length.

Framework region FW2 is preferably from 6 to 14 amino acids in length, more preferably from 8 to 12 amino acids in length. In certain preferred embodiments, FW2 is 12 amino acids in length. In other preferred embodiments, FW2 is 10 amino acids in length. In other preferred embodiments, FW2 is 9 amino acids in length. In other preferred embodiments, FW2 is 8 amino acids in length.

Hypervariable sequence HV2 is preferably from 4 to 11 amino acids in length, more preferably from to 10 amino acids in length. In certain preferred embodiments, HV2 is 10 amino acids in length. In certain preferred embodiments, HV2 is 9 amino acids in length. In other preferred embodiments, HV2 is 6 amino acids in length.

Framework region FW3a is preferably from 6 to 10 amino acids in length, more preferably from 7 to 9 amino acids in length. In certain preferred embodiments, FW3a is 8 amino acids in length. In certain preferred embodiments, FW3a is 7 amino acids in length.

Hypervariable sequence HV4 is preferably from 3 to 7 amino acids in length, more preferably from 4 to 6 amino acids in length. In certain preferred embodiments, HV4 is 5 amino acids in length. In other preferred embodiments, HV4 is 4 amino acids in length.

Framework region FW3b is preferably from 17 to 24 amino acids in length, more preferably from 18 to 23 amino acids in length, still more preferably from 19 to 22 amino acids in length. In certain preferred embodiments, FW3b is 21 amino acids in length. In other preferred embodiments, FW3b is amino acids in length.

CDR region CDR3 is preferably from 8 to 21 amino acids in length, more preferably from 9 to 20 amino acids in length, still more preferably from 10 to 19 amino acids in length. In certain preferred embodiments, CDR3 is 17 amino acids in length. In other preferred embodiments, CDR3 is 14 amino acids in length. In still other preferred embodiments, CDR3 is 12 amino acids in length. In yet other preferred embodiments, CDR3 is 10 amino acids in length.

Framework region FW4 is preferably from 7 to 14 amino acids in length, more preferably from 8 to 13 amino acids in length, still more preferably from 9 to 12 amino acids in length. In certain preferred embodiments, FW4 is 12 amino acids in length. In other preferred embodiments, FW4 is 11 amino acids in length. In still other preferred embodiments, FW4 is 10 amino acids in length. In yet other preferred embodiments, FW4 is 9 amino acids in length.

All possible combinations and permutations of the framework regions, complementarity determining regions and hypervariable regions listed above are explicitly contemplated herein.

Preferred VNAR domains for use in the present invention include B1, P3A1, D3, BA11, and E9, the sequences of which are set out below. B1, P3A1, D3 and E9 bind to ROR1 (data shown in co-pending International patent application no. PCT/EP2018/086823, published as WO 2019/122447, the content of which is incorporated herein by reference) PCT. BA11 is a humanised VNAR that binds with high affinity to human serum albumin (Kovalenko et al, J. Biol. Chem., 2013 JBC). In addition, the non-binding VNAR domain 2V is also described below.

B1 is
(SEQ ID NO: 44)
ASVNQTPRTATKETGESLTINCVVTGANYGLAATYWYRKNPGSSNQERI
SISGRYVESVNKRTMSFSLRIKDLTVADSATYYCKAYPWGAGAPWLVQW
YDGAGTVLTVN
2V is
(SEQ ID NO: 54)
TRVDQTPRTATKETGESLTINCVLTDTSYGLYSTSWFRKNPGTTDWERM
SIGGRYVESVNKGAKSFSLRIKDLTVADSATYYCKAQSLAISTRSYWYD
GAGTVLTVN
P3A1 is
(SEQ ID NO: 43)
TRVDQTPRTATKETGESLTINCVLTDTSYGLYSTSWFRKNPGTTDWERM
SIGGRYVESVNKGAKSFSLRIKDLTVADSATYYCKAREARHPWLRQWYD
GAGTVLTVN
D3 is
(SEQ ID NO: 39)
ASVNQTPRTATKETGESLTINCVLTDTSYGLYSTSWFRKNPGTTDWERM
SIGGRYVESVNKRAKSFSLRIKDLTVADSATYYCKAQSGMAISTGSGHG
YNWYDGAGTVLTVN
BA11 is
(SEQ ID NO: 55)
TRVDQSPSSLSASVGDRVTITCVLTDTSYPLYSTYWYRKNPGSSNKEQI
SISGRYSESVNKGTKSFTLTISSLQPEDSATYYCRAMSTNIWTGDGAGT
KVEIK
E9 is
(SEQ ID NO: 40)
AKVDQTPRTATKETGESLTINCVLTDTSYGLYSTSWFRKNPGTTDWERM
SIGGRYVESVNKRAKSFSLRIKDLTVADSATYYCKAQSGMAIDIGSGHG
YNWYDGAGTVLTVN

VNAR domains for use in the present invention include the VNARs identified in SEQ ID Nos: 40-49 and 59. Further preferred VNARs include the humanised VNARs identified in SEQ ID Nos: 45-49 and 59. A particularly preferred humanised VNAR is B1V15, having the amino acid sequence

(SEQ ID NO: 59)
ASVTQSPRSASKETGESLTITCRVTGANYGLAATYWYRKNPGSSNQERI
SISGRYSESVNKRTMSFSLRISSLTVEDSATYYCKAYPWGAGAPWLVQW
YDGQGTKLEVK.

Sequence identity referenced in relation to the molecules of the invention may be judged at the level of individual CDRs, HVs or FWs, or it may be judged over the length of the entire molecule. The CDR, HV and FW sequences described may also be longer or shorter, whether that be by addition or deletion of amino acids at the N- or C-terminal ends of the sequence or by insertion or deletion of amino acids with a sequence.

Any part of the specific binding protein may be engineered to enable conjugation in a PDC of the invention. In a preferred example, where an immunoglobulin Fc region is used, it may be engineered to include a cysteine residue as a conjugation site. Preferred introduced cysteine residues include, but are not limited to S252C and S473C (Kabat numbering), which correspond to S239C and S442C in EU numbering, respectively.

The target-binding molecule-drug conjugate may be any target-binding molecule-drug conjugate disclosed herein. For example, the target-binding molecule-drug conjugate may be selected from the group consisting of:
B1-hFc-vc-PAB-EDA-PNU
B1-hFc-va-EDA-PNU
B1-hFc-na-EDA-PNU

B1-hFc-vc-PAB-EDA-PNU and B1-hFc-va-EDA-PNU are shown herein to have particular efficacy for use in the treatment of mesothelioma and TNBC.

Preferably, target-binding molecule-drug conjugate comprises a PEG4 spacer. For example, the target-binding molecule-drug conjugate may be selected from the group consisting of:
B1-hFc-PEG4-vc-PAB-EDA-PNU
B1-hFc-PEG4-va-EDA-PNU
B1-hFc-PEG4-na-EDA-PNU

Preferably the hFc comprises an introduced cysteine residue at 5239C (EU numbering). Accordingly, for example, the target-binding molecule-drug conjugate may be selected from the group consisting of:
B1-hFc(S239C)-vc-PAB-EDA-PNU
B1-hFc(S239C)-va-EDA-PNU
B1-hFc(S239C)-na-EDA-PNU

The target-binding molecule-drug conjugate may be selected from the group consisting of:
B1-hFc(S239C)-PEG4-vc-PAB-EDA-PNU
B1-hFc(S239C)-PEG4-va-EDA-PNU
B1-hFc(S239C)-PEG4-na-EDA-PNU

In one embodiment of the target-binding molecule-drug conjugate the target-binding molecule is an antibody. In another embodiment of the target-binding molecule-drug conjugate the target-binding molecule binds HER-2. Preferably, the target-binding molecule is an antibody specific for HER-2.

Preferably, target-binding molecule-drug conjugate comprises a PEG4 spacer. For example, the target-binding molecule-drug conjugate may be selected from the group consisting of:
Tras-PEG4-vc-PAB-EDA-PNU
Tras-PEG4-va-EDA-PNU

The target-binding molecule-drug conjugate may be selected from the group consisting of:
Tras(S442C)-PEG4-vc-PAB-EDA-PNU
Tras(5442C)-PEG4-va-EDA-PNU

Any of the features described in respect of any of the above-mentioned aspects of the invention may be combined mutatis mutandis with the other aspects of the invention.

### Definitions

As used herein, an alkyl group is a straight chain or branched, substituted or unsubstituted group (preferably unsubstituted) containing from 1 to 40 carbon atoms. An alkyl group may optionally be substituted at any position. The term “alkenyl,” as used herein, denotes a group derived from the removal of a single hydrogen atom from a straight- or branched-chain aliphatic moiety having at least one carbon-carbon double bond. The term “alkynyl,” as used herein, refers to a group derived from the removal of a single hydrogen atom from a straight- or branched-chain aliphatic moiety having at least one carbon-carbon triple bond.

The term ‘alkyl’, ‘aryl’, ‘heteroaryl’ etc also include multivalent species, for example alkylene, arylene, ‘heteroarylene’ etc. Examples of alkylene groups include ethylene (—CH₂—CH₂—), and propylene (—CH₂—CH₂—CH₂—). An exemplary arylene group is phenylene (—C₆H₄—), and an exemplary heteroarylene group is pyridinylene (—C₅H₃N—).

Aromatic rings are cyclic aromatic groups that may have 0, 1, 2 or more, preferably 0, 1 or 2 ring heteroatoms. Aromatic rings may be optionally substituted and/or may be fused to one or more aromatic or non-aromatic rings (preferably aromatic), which may contain 0, 1, 2, or more ring heteroatoms, to form a polycyclic ring system.

Aromatic rings include both aryl and heteroaryl groups. Aryl and heteroaryl groups may be mononuclear, i.e. having only one aromatic ring (like for example phenyl or phenylene), or polynuclear, i.e. having two or more aromatic rings which may be fused (like for example napthyl or naphthylene), individually covalently linked (like for example biphenyl), and/or a combination of both fused and individually linked aromatic rings. Preferably the aryl or heteroaryl group is an aromatic group which is substantially conjugated over substantially the whole group. Aryl groups may contain from 5 to 40 ring carbon atoms, from 5 to 25 carbon atoms, from 5 to 20 carbon atoms, or from 5 to 12 carbon atoms. Heteroaryl groups may be from 5 to 40 membered, from 5 to 25 membered, from 5 to 20 membered or from 5 to 12 membered rings, containing 1 or more ring heteroatoms selected from N, O, S and P. An aryl or heteroaryl may be fused to one or more aromatic or non-aromatic rings (preferably an aromatic ring) to form a polycyclic ring system.

Aryl and heteroaryl preferably denote a mono-, bi- or tricyclic aromatic or heteroaromatic group with up to 25 ring atoms that may also comprise condensed rings and is optionally substituted. Preferred aryl groups include, without limitation, benzene, biphenylene, triphenylene, [1,1′:3′,1″ ]terphenyl-2′-ylene, naphthalene, anthracene, binaphthylene, phenanthrene, pyrene, dihydropyrene, chrysene, perylene, tetracene, pentacene, benzopyrene, fluorene, indene, indenofluorene, spirobifluorene, etc.

Preferred heteroaryl groups include, without limitation, 5-membered rings like pyrrole, pyrazole, silole, imidazole, 1,2,3-triazole, 1,2,4-triazole, tetrazole, furan, thiophene, selenophene, oxazole, isoxazole, 1,2-thiazole, 1,3-thiazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 6-membered rings like pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, and fused systems like carbazole, indole, isoindole, indolizine, indazole, benzimidazole, benzotriazole, purine, naphthimidazole, phenanthrimidazole, pyridimidazole, pyrazinimidazole, quinoxalinimidazole, benzoxazole, naphthoxazole, anthroxazole, phenanthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]thiophene, dithienothiophene, dithienopyridine, isobenzothiophene, dibenzothiophene, benzothiadiazothiophene, 2,5-dihydropyrrolo[3,4-c]pyrrol-1,4-dione (diketopyrrolopyrrole, DPP), 2-oxo-1H-indol-3-ylidene, [3,3′-bipyrrolo[2,3-b]pyridinylidene]-2,2′(1H,1′H)-dione (pyridine isoindigo) and (3E)-3-(2-oxo-1H-indol-3-ylidene)-1H-indol-2-one (isoindigo), or combinations thereof. The heteroaryl groups may be substituted with alkyl, alkoxy, thioalkyl, fluoro, fluoroalkyl or further aryl or heteroaryl substituents. Preferably a heteroaryl group is thiophene.

Particularly preferred heteroatoms are selected from O, S, N, P and Si. Typically, hydrogen will complete the valency of a heteroatom included in the molecules of the invention, e.g. for N there may be —NH— or —NH₂ where one or two other groups are involved.

As used herein, the term “optionally substituted” means that one or more of the hydrogen atoms in the optionally substituted moiety is replaced by a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable compounds. The term “stable”, as used herein, refers to compounds that are chemically feasible and can exist for long enough at room temperature (i.e. 16-25° C.) to allow for their detection, isolation and/or use in chemical synthesis.

Any of the above groups (for example, those referred to herein as “optionally substituted”, including alkyl, aryl and heteroaryl groups) may optionally comprise one or more substituents, preferably selected from silyl, sulfo, sulfonyl, formyl, amino, imino, nitrilo, mercapto, cyano, nitro, halogen, —NCO, —NCS, —OCN, —SCN, —C(═O)NR⁰R⁰⁰, —C(═O)X⁰, —C(═O)R⁰, —NR⁰R⁰⁰, C₁₋₁₂alkyl, C₁₋₁₂alkenyl, C₁₋₁₂alkynyl, C₆₋₁₂aryl, C₃₋₁₂cycloalkyl, heterocycloalkyl having 4 to 12 ring atoms, heteroaryl having 5 to 12 ring atoms, C₁₋₁₂ alkoxy, hydroxy, C₁₋₁₂ alkylcarbonyl, C₁₋₁₂ alkoxy-carbonyl, C₁₋₁₂ alkylcarbonyloxy or C₁₋₁₂ alkoxycarbonyloxy wherein one or more H atoms are optionally replaced by F or Cl and/or combinations thereof; wherein X⁰ is halogen and R⁰ and R⁰⁰ are, independently, H or optionally substituted C₁₋₁₂alkyl. The optional substituents may comprise all chemically possible combinations in the same group and/or a plurality of the aforementioned groups (for example amino and sulfonyl if directly attached to each other represent a sulfamoyl radical). In one embodiment, the substituent is not acyl. As used herein acyl refers to an acyl group which is a moiety derived by the removal of one or more hydroxyl groups from an oxoacid, such as a carboxylic acid. It contains a double-bonded oxygen atom and an alkyl group.

In some embodiments the groups may be unsubstituted. For example, in respect of the first aspect of the invention, the anthracycline (PNU) derivative may be of formula (I):

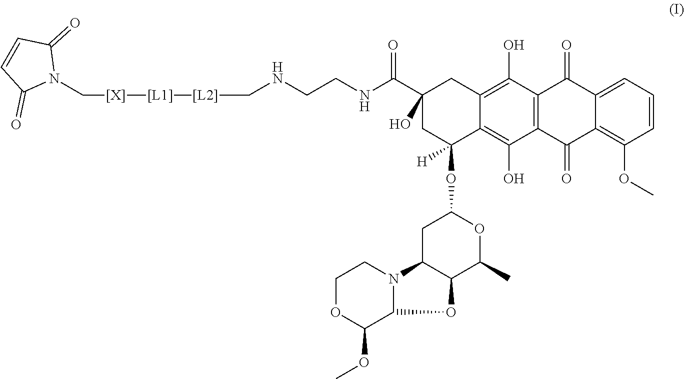

wherein [X] is an optional spacer selected from the group comprising unsubstituted alkyl groups, unsubstituted heteroalkyl groups, unsubstituted aryl groups, unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, any amino acid except glycine, and combinations thereof.

In embodiments wherein the groups are unsubstituted, [X] is preferably selected from the group comprising polyethylene glycol and

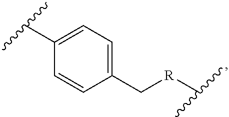

wherein [symbol] represents the point of attachment to the rest of the molecule and wherein [R] is an optional spacer selected from the group comprising unsubstituted alkyl groups, unsubstituted heteroalkyl groups, unsubstituted aryl groups, unsubstituted heteroaryl groups, one or more heteroatoms, polyethylene glycol, or a combination thereof.

In general, the term PAB is intended to mean p-aminobenzyloxycarbonyl. Occasionally in the literature, the term PAB may be used to indicated p-aminobenzyl. In the present specification, PAB is intended to indicate p-aminobenzyloxycarbonyl.

The term “protein” means, in general terms, a plurality of amino acid residues joined together by peptide bonds. It is used interchangeably and means the same as peptide, oligopeptide, oligomer or polypeptide, and includes glycoproteins and derivatives thereof. The term “protein” is also intended to include fragments, analogues, variants and derivatives of a protein wherein the fragment, analogue, variant or derivative retains essentially the same biological activity or function as a reference protein. Examples of protein analogues and derivatives include peptide nucleic acids, and DARPins (Designed Ankyrin Repeat Proteins).

The term “target-binding molecule” refers to any molecule that binds to a given target. In this context, “target” and “antigen” may be used interchangeably. Examples of target-binding molecules include natural or recombinant proteins including immunoglobulins or antibodies, immunoglobulin Fc regions, immunoglobulin Fab regions, Fab, Fab′, Fv, Fv-Fc, single chain Fv (scFv), scFv-Fc, (scFv)₂, diabodies, triabodies, tetrabodies, bispecific t-cell engagers (BiTEs), inteins, intein fusions, VNAR domains, single domain antibodies (sdAb), VH domains, scaffold proteins (affibodies, centyrins, darpins etc.) and nucleic acids including aptamers or small molecules or natural products that have been developed to bind to the target or naturally bind to the target.

An antigen specific binding protein may comprise any protein which binds to a given antigen. Preferred examples include an immunoglobulin or antibody, an immunoglobulin Fc region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager (BiTE), an intein, an intein fusion, a VNAR domain, a single domain antibody (sdAb), a VH domain, or a scaffold protein (affibodies, centyrins, darpins etc.). Particularly preferred examples include VNAR domains that comprise an amino acid sequence derived from a synthetic library of VNAR molecules, or from libraries derived from the immunization of a cartilaginous fish. The terms VNAR, IgNAR and NAR may be used interchangeably also.

Amino acids are represented herein as either a single letter code or as the three letter code or both. Chemical modification of proteins and biomolecules to introduce thiols is well established. Methods include reaction of amine groups with 2-iminothiolane (Traut's reagent), modification of amine groups with NHS-ester containing heterobifunctional agents such as N-succinimidyl S-acetylthiolate (SATA) or N-succinimidyl-4-(2-pyridyldithio)butanoate (SPDB), followed by treatment with hydroxylamine and reducing agents respectively and cleavage of engineered intein-fusion proteins with cysteamine to generate C-terminal thiol proteins and peptides.

The term “affinity purification” means the purification of a molecule based on a specific attraction or binding of the molecule to a chemical or binding partner to form a combination or complex which allows the molecule to be separated from impurities while remaining bound or attracted to the partner moiety.

The term “Complementarity Determining Regions” or CDRs (i.e., CDR1 and CDR3) refers to the amino acid residues of a VNAR domain the presence of which are typically involved in antigen binding. Each VNAR typically has two CDR regions identified as CDR1 and CDR3. Additionally, each VNAR domain comprises amino acids from a “hypervariable loop” (HV), which may also be involved in antigen binding. In some instances, a complementarity determining region can include amino acids from both a CDR region and a hypervariable loop. In other instances, antigen binding may only involve residues from a single CDR or HV. According to the generally accepted nomenclature for VNAR molecules, a CDR2 region is not present.

“Framework regions” (FW) are those VNAR residues other than the CDR residues. Each VNAR typically has five framework regions identified as FW1, FW2, FW3a, FW3b and FW4.

The boundaries between FW, CDR and HV regions in VNARs are not intended to be fixed and accordingly some variation in the lengths and compositions of these regions is to be expected. This will be understood by those skilled in the art, particularly with reference to work that has been carried out in analyzing these regions. (Anderson et al., PLoS ONE (2016) 11 (8); Lui et al., Mol Immun (2014) 59, 194-199; Zielonka et al., Mar Biotechnol (2015). 17, (4) 386-392; Fennell et al., J Mol Biol (2010) 400. 155-170; Kovalenko et al., J Biol Chem (2013) 288. 17408-17419; Dooley et al., (2006) PNAS 103 (6). 1846-1851). The molecules of the present invention, although defined by reference to FW, CDR and HV regions herein, are not limited to these strict definitions. Variation in line with the understanding in the art as the structure of the VNAR domain is therefore expressly contemplated herein.

A “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein.

A codon set is typically represented by 3 capital letters in italics, e.g. NNK, NNS, XYZ, DVK etc. A “non-random codon set” therefore refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et al.; J. Mol. Biol. (1999), 296, 57-86); Garrard & Henner, Gene (1993), 128, 103). Such sets of oligonucleotides having certain codon sets can be synthesized using commercial nucleic acid synthesizers (available from, for example, Applied Biosystems, Foster City, Calif.), or can be obtained commercially (for example, from Life Technologies, Rockville, Md.). A set of oligonucleotides synthesized having a particular codon set will typically include a plurality of oligonucleotides with different sequences, the differences established by the codon set within the overall sequence. Oligonucleotides used according to the present invention have sequences that allow for hybridization to a VNAR nucleic acid template and also may where convenient include restriction enzyme sites.

“Cell”, “cell line”, and “cell culture” are used interchangeably (unless the context indicates otherwise) and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

The “detection limit” for a chemical entity in a particular assay is the minimum concentration of that entity which can be detected above the background level for that assay. For example, in the phage ELISA, the “detection limit” for a particular phage displaying a particular antigen binding fragment is the phage concentration at which the particular phage produces an ELISA signal above that produced by a control phage not displaying the antigen binding fragment.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

The term “fusion protein” in this text means, in general terms, one or more proteins joined together by chemical means, including hydrogen bonds or salt bridges, or by peptide bonds through protein synthesis or both. Typically fusion proteins will be prepared by DNA recombination techniques and may be referred to herein as recombinant fusion proteins.

“Identity” describes the relationship between two or more polypeptide sequences or two or more polynucleotide sequences, as determined by comparing the sequences. Identity also means the degree of sequence relatedness (homology) between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic acids Research, 12, 387 (1984), BLASTP, BLASTN, and FASTA (Atschul et al., J. Molec. Biol. (1990) 215, 403).

Preferably, the amino acid sequence of the protein has at least 45% identity, using the default parameters of the BLAST computer program (Atschul et al., J. Mol. Biol. (1990) 215, 403-410) provided by HGMP (Human Genome Mapping Project), at the amino acid level, to the amino acid sequences disclosed herein.

More preferably, the protein sequence may have at least 45%, 46%, 47%, 48%, 49%, 50%, 55%, 60%, 65%, 66%, 67%, 68%, 69%, 70%, 75%, 80%, 85%, 90% and still more preferably 95% (still more preferably at least 96%, 97%, 98% or 99%) identity, at the nucleic acid or amino acid level, to the amino acid sequences as shown herein.

The protein may also comprise a sequence which has at least 45%, 46%, 47%, 48%, 49%, 50%, 50%, 55%, 60%, 65%, 66%, 67%, 68%, 69%, 70%, 75%, 80%, 85%, 90%, 95%, 96%, 97%, 98%, or 99% identity with a sequence disclosed herein, using the default parameters of the BLAST computer program provided by HGMP, thereto

A “mutation” is a deletion, insertion, or substitution of a nucleotide(s) relative to a reference nucleotide sequence, such as a wild type sequence.

“Natural” or “naturally occurring” VNARs, refers to VNARs identified from a non-synthetic source, for example, from a tissue source obtained ex vivo, or from the serum of an animal of the Elasmobranchii subclass. These VNARs can include VNARs generated in any type of immune response, either natural or otherwise induced. Natural VNARs include the amino acid sequences, and the nucleotide sequences that constitute or encode these antibodies. As used herein, natural VNARs are different than “synthetic VNARs”, synthetic VNARs referring to VNAR sequences that have been changed from a source or template sequence, for example, by the replacement, deletion, or addition, of an amino acid, or more than one amino acid, at a certain position with a different amino acid, the different amino acid providing an antibody sequence different from the source antibody sequence.

A fragment, analogue, variant or derivative of the protein may be at least 25 preferably 30 or 40, or up to 50 or 100, or 60 to 120 amino acids long, depending on the length of the original protein sequence from which it is derived. A length of 90 to 120, 100 to 110 amino acids may be convenient in some instances.

The fragment, derivative, variant or analogue of the protein may be (i) one in which one or more of the amino acid residues are substituted with a conserved or non-conserved amino acid residue (preferably, a conserved amino acid residue) and such substituted amino acid residue may or may not be one encoded by the genetic code, or (ii) one in which one or more of the amino acid residues includes a substituent group, or (iii) one in which the additional amino acids are fused to the mature polypeptide, such as a leader or auxiliary sequence which is employed for purification of the polypeptide. Such fragments, derivatives, variants and analogues are deemed to be within the scope of those skilled in the art from the teachings herein.

“Oligonucleotides” are short-length, single- or double-stranded polydeoxynucleotides that are chemically synthesized by known methods (such as phosphotriester, phosphite, or phosphoramidite chemistry, using solid-phase techniques). Further methods include the polymerase chain reaction (PCR) used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides can be purified on polyacrylamide gels or molecular sizing columns or by precipitation. DNA is “purified” when the DNA is separated from non-nucleic acid impurities (which may be polar, non-polar, ionic, etc.).

A “variant” or “mutant” of a starting or reference polypeptide (for example, a source VNAR or a CDR thereof), such as a fusion protein (polypeptide) or a heterologous polypeptide (heterologous to a phage), is a polypeptide that (1) has an amino acid sequence different from that of the starting or reference polypeptide and (2) was derived from the starting or reference polypeptide through either natural or artificial mutagenesis. Such variants include, for example, deletions from, and/or insertions into and/or substitutions of, residues within the amino acid sequence of the polypeptide of interest. For example, a fusion polypeptide of the invention generated using an oligonucleotide comprising a nonrandom codon set that encodes a sequence with a variant amino acid (with respect to the amino acid found at the corresponding position in a source VNAR or antigen binding fragment) would be a variant polypeptide with respect to a source VNAR or antigen binding fragment. Thus, a variant CDR refers to a CDR comprising a variant sequence with respect to a starting or reference polypeptide sequence (such as that of a source VNAR or antigen binding fragment). A variant amino acid, in this context, refers to an amino acid different from the amino acid at the corresponding position in a starting or reference polypeptide sequence (such as that of a source VNAR or antigen binding fragment). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

A “wild-type” or “reference” sequence or the sequence of a “wild-type” or “reference” protein/polypeptide, such as a coat protein, or a CDR of a source VNAR, may be the reference sequence from which variant polypeptides are derived through the introduction of mutations. In general, the “wild-type” sequence for a given protein is the sequence that is most common in nature. Similarly, a “wild-type” gene sequence is the sequence for that gene which is most commonly found in nature. Mutations may be introduced into a “wild-type” gene (and thus the protein it encodes) either through natural processes or through man induced means. The products of such processes are “variant” or “mutant” forms of the original “wild-type” protein or gene.

The term “conjugation” as used herein may refer to any method of chemically linking two or more chemical moieties. Typically, conjugation will be via covalent bond. In the context of the present invention, at least one of the chemical moieties will be a target-binding molecule and another or the molecules will be the PNU derivative of the invention. In some cases the conjugation will involve two or more target-binging molecules in addition to a PNU derivative of the invention, in which case conjugation may be directly between the target-binding molecules with the PNU derivative conjugated to one of the target-binding molecules.

The phrase “selected from the group comprising” may be substituted with the phrase “selected from the group consisting of” and vice versa, wherever they occur herein.

The present invention will be further understood by reference to the following examples.

### EXAMPLES

### Example 1: Characterisation of PNU Derivative

The PNU derivatives of the invention were prepared accordingly to standard synthesis methods. Mass spectrometry was used to verify that the correct molecules had been produced (table 1).

TABLE 1
Characterisation of PNU derivatives by mass spectrometry

| | | Exp. | Obs. |
|---|---|---|---|
| | | mass, | mass, |
| Name | Structure | Da | Da |
| MA-PEG4- vc-PAB- EDA- PNU159682 | 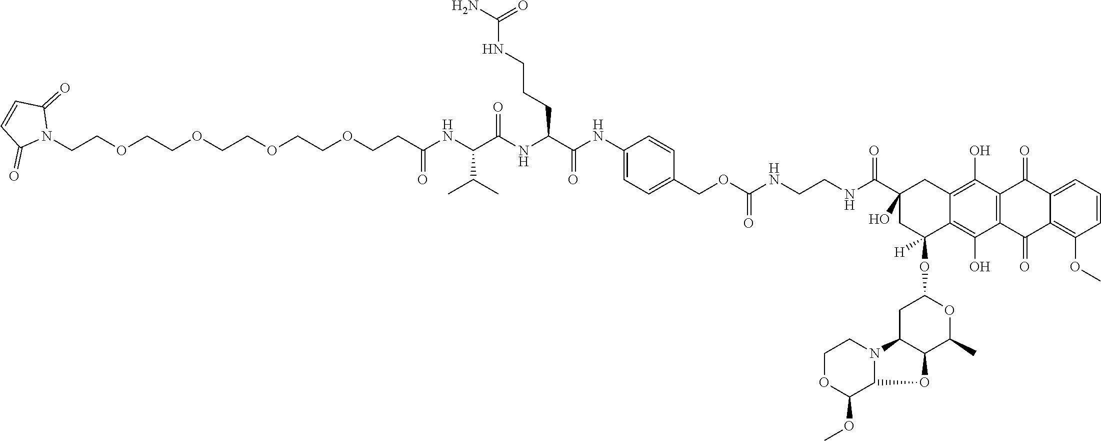 | 1402.5 | 1402.1 |
| MA-PEG4- va-EDA- PNU159682 | 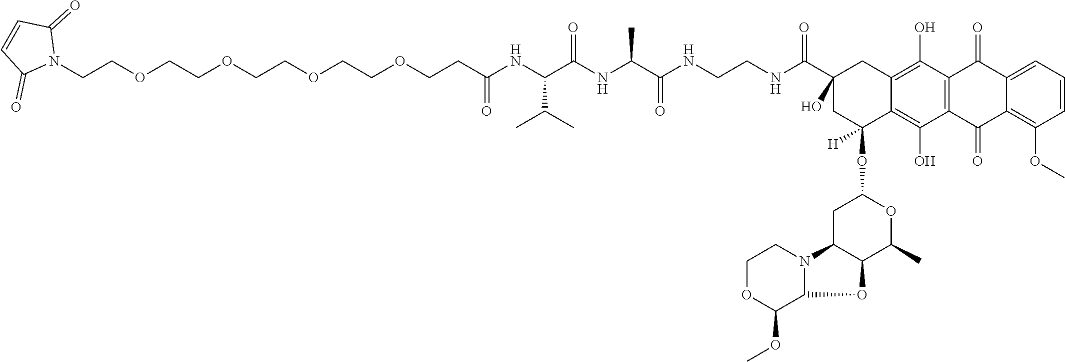 | 1167.2 | 1167.6 |
| MA-PEG4- na-EDA- PNU159682 | 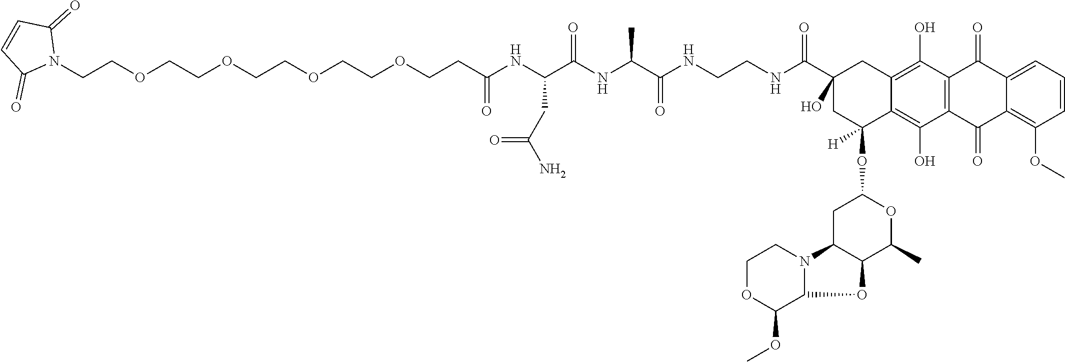 | 1182.2 | 1182.4 |
| MA-PEG4- EDA- PNU159682 | 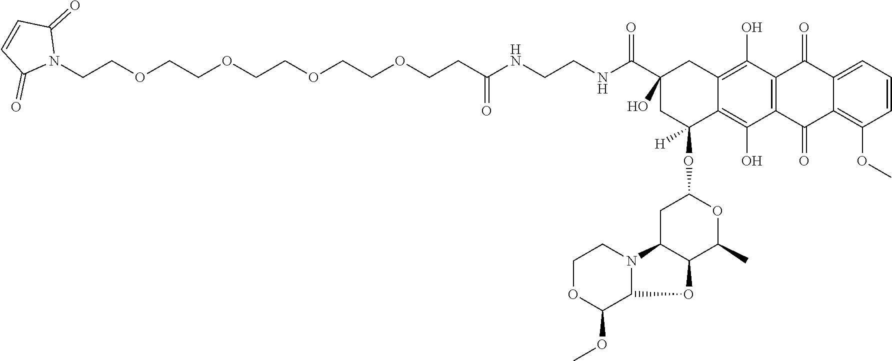 | 997.01 | 996.8 |
| MA-PEG4- vc-PAB- DMAE- PNU159682 | 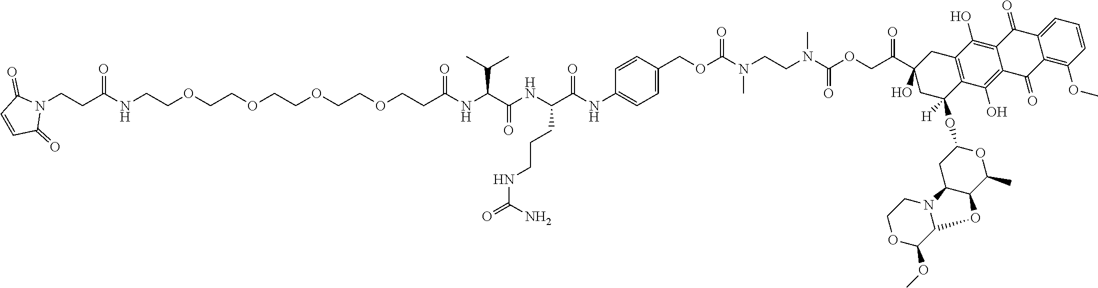 | 1559.6 | 1558.7 |

The potency of the EDA-PNU159682 derivative (FIG. 1) was tested in a variety of cell lines. The results are summarised in Table 2.

TABLE 2
EDA PNU159682 derivative free payload IC50s over 96 h

| | | IC50 (pM) | |
|---|---|---|---|
| | | | EDA -PNU159682 |
| Cell line | Tissue | PNU159682 | derivative |
| PA-1 | Ovarian | 4.4 | 19 |
| PA-1 32.35 | Ovarian ROR1 −/− | 1.4 | 4.2 |
| PC-9 | Lung | 47 | 96 |
| NCI-N87 | Stomach | 122 | 190 |
| DU145 | Prostate | 22 | 790 |
| MDA-231 | Breast | 11 | 29 |
| MHH-ES1 | Ewings sarcoma | 3.4 | 17 |
| A427 | Lung | 9.8 | 31 |
| Panc-1 | Pancreatic | 26 | 54 |
| A549 | Lung | 13 | 15 |
| ACHN | Kidney | 9.7 | 175 |
| Hs746T | Stomach | 49 | 28 |
| Mino | MCL | 37 | 57 |
| 697 | BCL | 8 | 24 |
| JeKo-1 | MCL | 44 | 330 |
| MDA-468 | Breast | 21 | 59 |
| PA-1 31-J-06 | Ovarian ROR1 −/− | 2.2 | 12 |
| Kasumi-2 | BCL | 2.4 | 21 |

### Example 2: VNAR-hFc PNU Conjugates

A number of VNAR-hFc-PNU conjugates were prepared in order to investigate the PNU derivatives of the invention. Two VNARs were specific for ROR1 (B1 and P3A1). In addition, a non-binding VNAR (2V) was used as a control molecule.

VNARs were genetically fused to engineered hIgG1 Fc domains via standard [G₄S]₃ that contained a cysteine substitution in the hIgG1 Fc sequence, S239C (EU numbering). The VNAR Fc fusion proteins were expressed as secreted protein in CHO K1 cells and purified from the media using MabSelect™ SuRe™ (Evitria, Switzerland). Purified proteins were analysed by SEC (AdvanceBio, Agilent), SDS PAGE and mass spectrometry to confirm sequence and protein integrity. Binding kinetics were determined using a Pioneer Surface Plasmon Resonance (SPR) instrument (SensiQ/Pall ForteBio), or the Biolayer Interferometry (BLI) Octet K2 system (ForteBio). ROR1-hFc or ROR2-hFc fusion proteins (extracelluar domains) were immobilised in sodium acetate pH5 buffer to COOH₂ chips or AR2G sensors using amine coupling. VNARs and VNAR-Fc molecules were tested at various concentrations and the Ka (M⁻¹ s⁻¹), Kd (s⁻¹) and KD (nM) values were determined using QDat software (SensiQ/Pall ForteBio) or Octet Data Analysis High Throughput software (ForteBio) for Biolayer Interferometry. ROR1 2A2 mAb (Biolegend) and ROR2 mAb (R&D Systems) were included as controls for positive/negative binding to ROR1 and ROR2. 2V is a control VNAR sequence, derived from a naïve VNAR library, so is representative of this protein class but has no known target. Tables 3 and 3b summarise the surface plasmon resonance data for the affinity of these molecules for human ROR1 & human ROR2.

TABLE 3
SPR data for binding of VNAR-Fc fusions
to human ROR1 and human ROR2

| | hROR1 | | | |
|---|---|---|---|---|
| Molecule | Ka (M⁻¹s⁻¹) | Kd (s⁻¹) | KD (nM) | hROR2 |
| B1 hFc | 3.08E+06 | 9.53E−05 | 0.032 | No binding |
| P3A1 hFc | 1.07E+07 | 5.64E−04 | 0.084 | No binding |
| D3 hFc | 1.21E+06 | 2.88E−03 | 2.6 | No binding |
| E9 hFc | 7.07E+05 | 3.64E−03 | 5.3 | No binding |
| D3-D3 hFc | 4.96E+06 | 9.88E−04 | 0.25 | No binding |
| hFc - P3A1 | 2.38E+06 | 7.76E−04 | 0.35 | No binding |
| hFc - D3 | 1.10E+06 | 2.35E−03 | 2.37 | No binding |
| hFc - D3-D3 | 2.35E+06 | 1.01E−03 | 0.49 | No binding |
| 2V hFc | No binding | No binding | No binding | No binding |
| 2V-2V hFc | No binding | No binding | No binding | No binding |

The same process was repeated for P3A1 hFc(442), resulting in comparable binding data. For this derivative the VNAR was genetically fused to engineered hIgG1 Fc domains that contained a cysteine substitution in the hIgG1 Fc sequence, S442C (EU numbering). Surface plasmon resonance data for the affinity of this molecule for human ROR1 & human ROR2 is shown in Table 3b.

TABLE 3b
SPR data for binding of VNAR-Fc fusions
to human ROR1 and human ROR2

| | hROR1 | | | |
|---|---|---|---|---|
| Molecule | Ka (M⁻¹s⁻¹) | Kd (s⁻¹) | KD (nM) | hROR2 |
| B1V15 hFc | 1.79E+5 | 1.3E−04 | 0.726 | No binding |
| P3A1 | 2.70+05 | 2.0E−04 | 0.741 | No binding |
| hFc(442) | | | | |

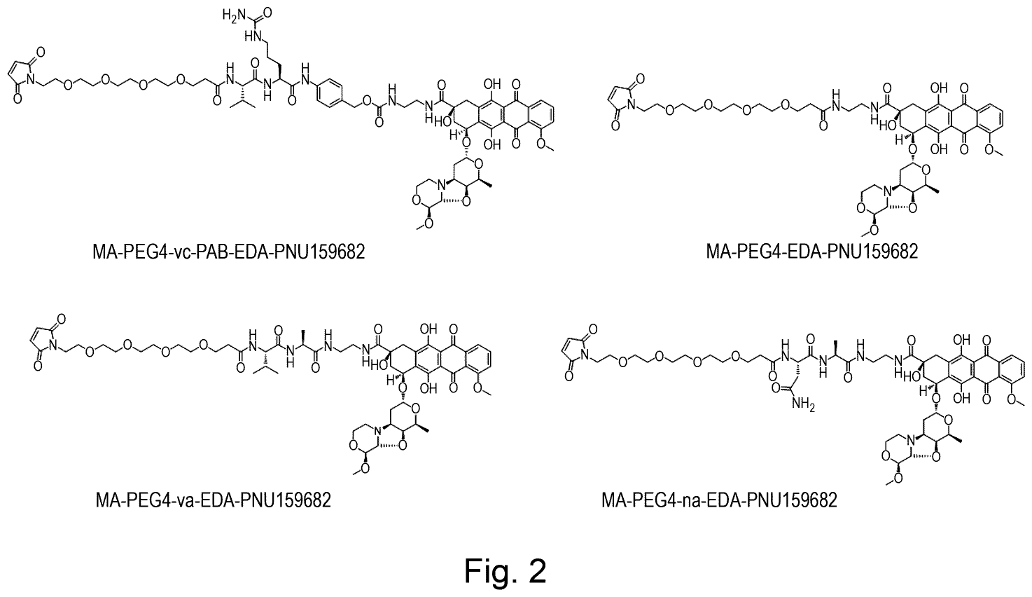

Using a partial reduction, refolding and labelling method adapted from the literature [Junutula et al, 2008 Nat Biotech, Jeffrey et al, 2013 Bioconj Chem], these proteins were site specific labelled with the maleimide PNU derivatives (FIGS. 1 and 2). Briefly, 1 mg/mi VNAR hFc solutions were prepared in PBS+100 mM L-Arginine pH7.4 with 1 mM EDTA. 20 molar equivalents TCEP added and incubated at 4° C. for a minimum of 48 hours. 30 molar equivalents DHAA added, pH adjusted to 6.5 and incubated at room temperature for 1 hour. Refolded VNAR Fc S239C was extensively dialysed or buffer exchanged into PBS+50 mM L-Arginine and quantified by UV before reacting with 4 or 5 molar equivalents maleimide PNU solution, room temperature overnight. Conjugates were purified by SEC and analysed by analytical HIC, analytical SEC, and LC-MS. Table 4 summaries the conjugates prepared.

TABLE 4
Summary of characteristics of VNAR-PNU conjugates

| | | Deglycosylated, reduced | | | Conjugate | HMW |
|---|---|---|---|---|---|---|
| | | mass, Da | | DAR by | Yield | aggregate |
| Protein | Payload | Expected | Observed | MS | % | % |
| B1 hFc | MA-PEG4-vc-PAB-EDA-PNU159682 | 40,262.2 | 40,263.3 | 2.1 | 46 | <1 |
| | MA-PEG4-va-EDA-PNU159682 | 40,026.9 | 40,027.6 | 2.0 | 50 | <1 |
| | MA-PEG4-na-EDA-PNU159682 | 40,041.9 | 40,043.2 | 2.0 | 59 | <1 |
| | MA-PEG4-EDA-PNU159682 | 39,856.7 | 39,857.6 | 2.0 | 53 | <1 |
| | MA-PEG4-vc-PAB-DMAE-PNU159682 | 40,419.2 | 40,421.9 | 2.2 | 16 | 0.4 |
| P3A1 hFc | MA-PEG4-vc-PAB-EDA-PNU159682 | 40,400.3 | 40,402.6 | 2.2 | 60 | <1 |
| | MA-PEG4-va-EDA-PNU159682 | 40,165.0 | 40167.4 | 2.1 | 55 | <1 |
| | MA-PEG4-vc-PAB-DMAE-PNU159682 | 40,557.4 | 40559.6 | 2.0 | 70 | <1 |
| 2V hFc | MA-PEG4-vc-PAB-EDA-PNU159682 | 40,177.0 | 40,180.6 | 2.1 | 70 | <1 |
| | MA-PEG4-va-EDA-PNU159682 | 39,941.7 | 39,942.4 | 2.0 | 69 | <1 |
| | MA-PEG4-na-EDA-PNU159682 | 39,956.7 | 39,957.5 | 2.0 | 65 | <1 |
| | MA-PEG4-vc-PAB-DMAE-PNU159682 | 40,334.1 | 40,337.0 | 2.4 | 55 | 0 |

The same process was repeated for the P3A1 hFc(442)-va-EDA-PNU conjugate. Table 4b summaries the conjugate prepared.

| | | Deglycosylated, reduced | | | Conjugate | HMW |
|---|---|---|---|---|---|---|
| | | mass, Da | | DAR | Yield | aggregate |
| Protein | Payload | Expected | Observed | by MS | % | % |
| P3A1 | MA-PEG4-va-EDA-PNU159682 | 40,163.8 | 40,164.9 | 1.4 | 14 | <1 |
| hFc(442) | | | | | | |

In Vitro Cell Viability Assays for Cancer Cells Treated with Anti ROR1 VNAR Drug Conjugates

Cells were seeded into white, clear bottom 96 well plates (Costar) and incubated at 37° C., 5% CO₂ for 24 hours. On the following day, dilution series were set up for each test agent at ×10 working stocks. The dose response ×10 stock was: 10000, 5000, 1000, 500, 100, 50, 10, 5, 1, 0.5 nM etc. 10 μL of the ×10 stock solutions were added to the cell plates (90 μl per well) using a multichannel pipette. This resulted in a 1:10 dilution into the well and dose responses ranging from 1000 nM (column 1) to 0.05 nM (column 10) or continued to 0.5 fM, if required, for the most sensitive cells lines. 100d of vehicle control (PBS) was added to the control wells (columns 11 and 12). Plates were incubated at 37′C, 5% CO₂ for 72-96 hours. Promega Cell litre Glo reagent was used as per the manufacturer's instructions to assess cell viability. Briefly, assay plates were removed from incubator and allowed to equilibrate to room temperature before adding 1000d of room temperature Cell litre Glo reagent to each 100 μl assay well. Plates were placed on a plate shaker for 2 minutes at 600 rpm. Plates were allowed to sit for a further 10 minutes at room temperature prior to measuring luminescence read-out using a Clariostar plate-reader (BMG). Data was analysed by calculating the average for untreated (vehicle only) control wells and determining the % of control for each treated well. % of control data was then plotted against Log [Treatment] concentration and the IC50 value derived using non-linear regression fitting in GraphPad Prism software.

The following cell lines were used:
Kasumi-2—human B cell leukaemia precursor;
PA-1—human ovarian cancer cell line;
PA-1 ROR1 ko—human ovarian cancer cell line with ROR1 knock-out;
697—human B cell leukaemia precursor, and
MHH-ES1—human Ewing's sarcoma cell line.

TABLE 5
In vitro cell killing data for VNAR-hFc-PNU conjugates

| | | | | IC50(nM) 96 h | | | | |
|---|---|---|---|---|---|---|---|---|
| | | | | | | PA-1 ROR1 | | |
| Molecule | Protein | Payload | DAR | Kasumi-2 | PA-1 | ko | 697 | MHH-ES1 |
| P3A1hFc-PEG4-vc-PAB-EDA-PNU | P3A1-hFc | PEG4-vc-PAB-EDA-PNU159682 | 2.2 | 3.2 | 0.1 | 11 | 52 | 24 |
| P3A1hFc-PEG4-va-EDA-PNU | P3A1-hFc | PEG4-va-EDA-PNU159682 | 2.1 | 2.3 | 0.15 | 11.3 | 61 | 18 |
| B1hFc-PEG4-vc-PAB-EDA-PNU | B1-hFc | PEG4-vc-PAB-EDA-PNU159682 | 2 | 7 | 0.1 | 4.7 | 40 | 3 |
| B1hFc-PEG4-va-EDA-PNU | B1-hFc | PEG4-va-EDA-PNU159682 | 2 | 0.093 | 0.0016 | 10.3 | 68 | 4.6 |
| B1hFc-PEG4-na-EDA-PNU | B1-hFc | PEG4-na-EDA-PNU159682 | 2 | 0.016 | 0.0064 | 9.2 | 7.7 | 1.1 |
| B1hFc-PEG4-EDA-PNU | B1-hFc | PEG4-EDA-PNU159682 | 2 | 0.014 | 0.18 | 34 | 15 | 4.5 |
| 2VhFc-PEG4-vc-PAB-EDA-PNU | 2V-hFc | PEG4-vc-PAB-EDA-PNU159682 | 2 | 58 | 7.3 | 14 | 63 | 26 |
| 2VhFc-PEG4-va-EDA-PNU | 2V-hFc | PEG4-va-EDA-PNU159682 | 2 | 98 | 54 | 48 | 256 | 152 |
| 2VhFc-PEG4-na-EDA-PNU | 2V-hFc | PEG4-na-EDA-PNU159682 | 2 | 45 | 47.5 | 35 | 108 | 50 |
| 2VhFc-PEG4-vc-PAB-DMAE-PNU | 2V-hFc | PEG4-vc-PAB-DMAE-PNU159682 | 2 | 11 | 3.13 | 4.5 | 40 | 4.7 |
| P3A1hFc(442)-PEG4-va-EDA-PNU | P3A1-hFc | PEG4-va-EDA-PNU159682 | 1.4 | 9.1 | 0.025 | 18.3 | | 37.5 |

TABLE 6
Comparison of ROR1-specific conjugates
to non-binding 2V-hFc conjugate

| | ROR1 targeting/2V non-targeting window | | | | |
|---|---|---|---|---|---|
| | | | PA-1 | | MHH- |
| Molecule | Kasumi-2 | PA-1 | 31-J-06 | 697 | ES1 |
| P3A1hFc-PEG4-vc-PAB- | 18 | 73 | 1.3 | 1.2 | 1.1 |
| EDA-PNU | | | | | |
| P3A1hFc-PEG4-va- | 43 | 360 | 4 | 4 | 8.4 |
| EDA-PNU | | | | | |
| B1hFc-PEG4-vc-PAB- | 8 | 73 | 3 | 1.6 | 9 |
| EDA-PNU | | | | | |
| B1hFc-PEG4-va-EDA- | 1043 | 33750 | 5 | 3.8 | 33 |
| PNU | | | | | |

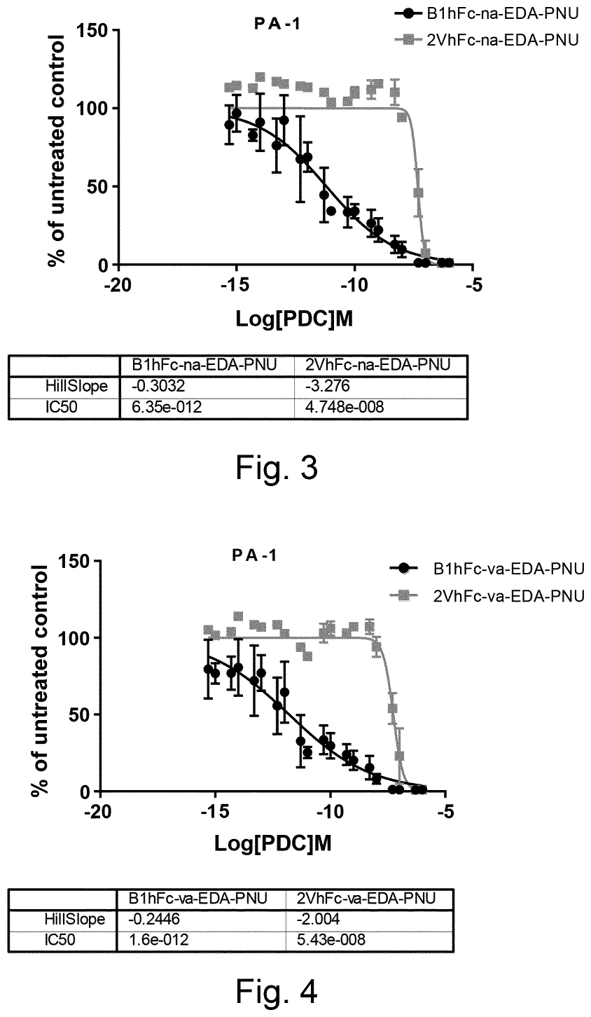

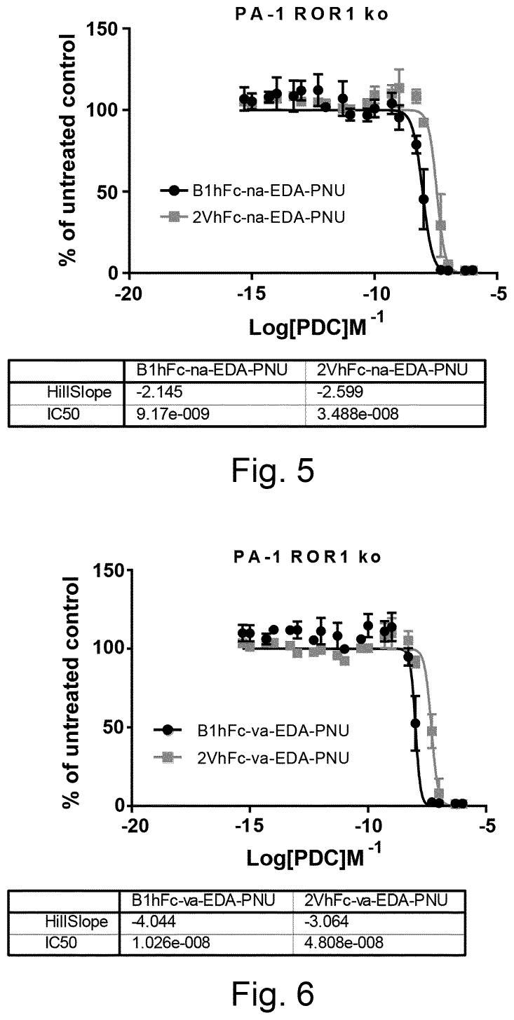

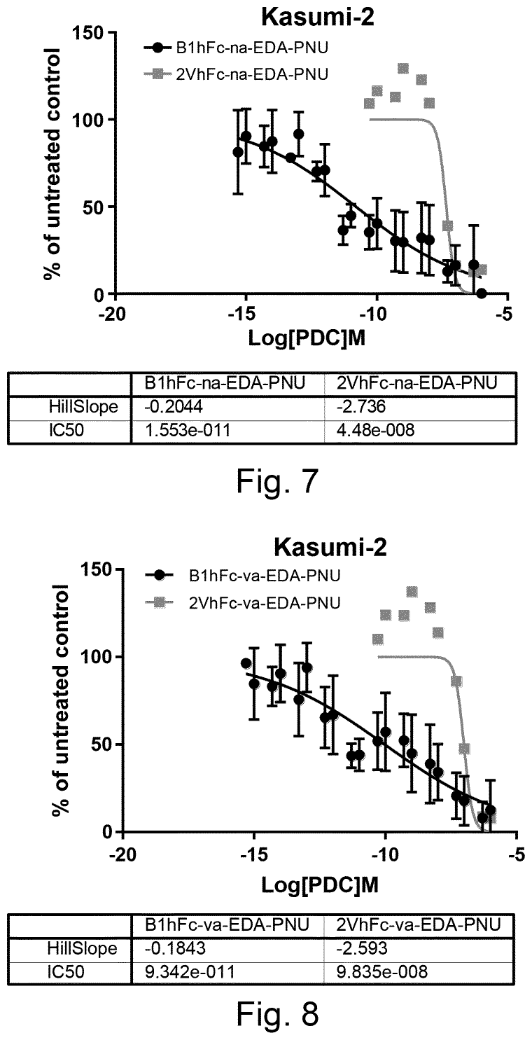

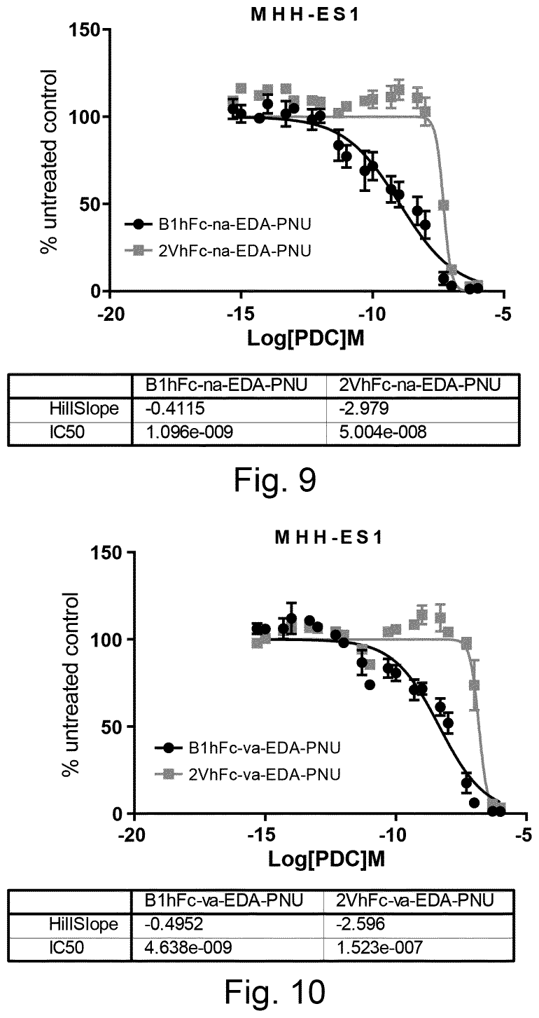

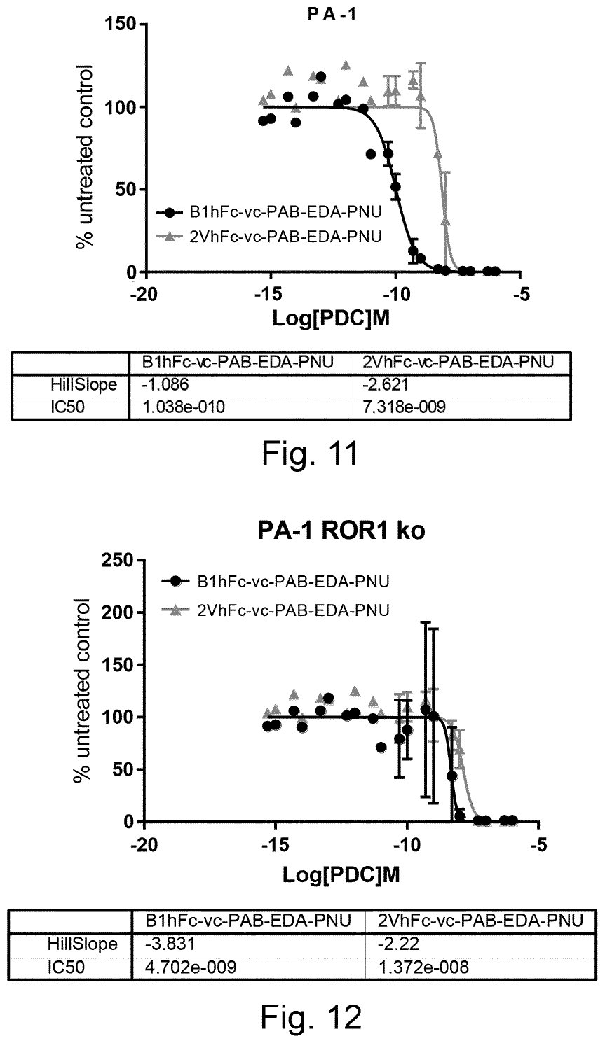

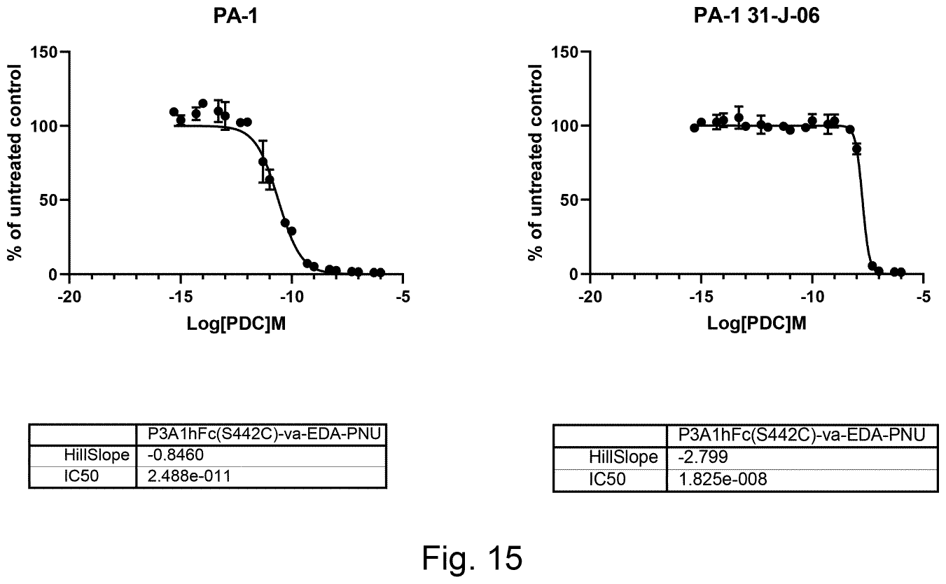

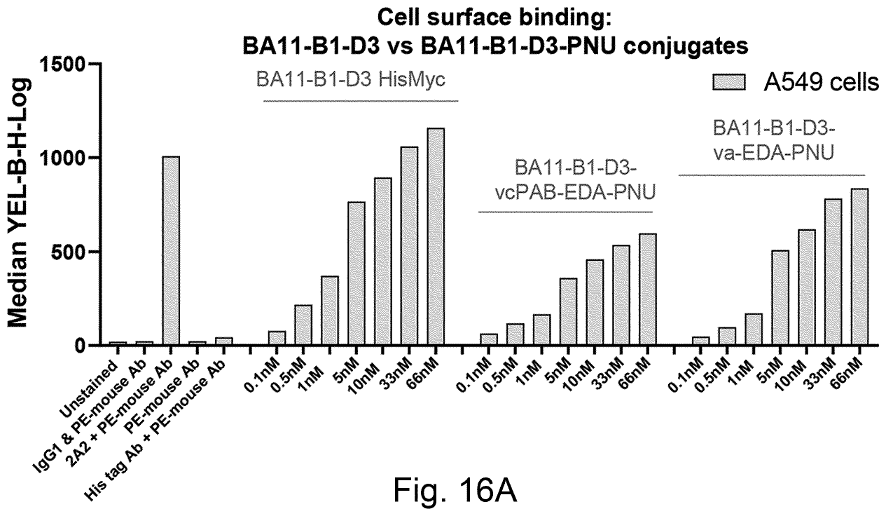

Tables 5 and 6 and FIGS. 3 to 12 and FIG. 15 show that ROR1 targeting protein drug conjugates using payloads of the invention are highly potent at killing ROR9 expressing cancer cells in an ROR1 dependent fashion, with large windows when compared to corresponding non-binding protein drug conjugates (2V hFc).

TABLE 7
Potency of non-binding VNAR(2V)-hFc - PNU159682 conjugates
2V is a control VNAR sequence, derived from a naïve VNAR library,
so has no known target and doesn't bind to cancer cell-lines by
flow cytometry. 2V-hFc-PNU conjugates were generated and assessed
for non-selective cell-killing using a panel of cancer cell-lines
as previously described.

| | | | IC50(nM)96 h | | | | | |
|---|---|---|---|---|---|---|---|---|
| | | | | | PA-1 | | MHH- | |
| Molecule | Payload | DAR | Kasumi-2 | PA-1 | ROR1 ko | 697 | ES1 | ACHN |
| 2VhFc-PEG4-vc-PAB-EDA-PNU | PEG4-vc-PAB-EDA-PNU159682 | 2 | 58 | 7.3 | 14 | 63 | 26 | 93 |
| 2VhFc-PEG4-va-EDA-PNU | PEG4-va-EDA-PNU159682 | 2 | 98 | 54 | 48 | 256 | 152 | 483 |
| 2VhFc-PEG4-na-EDA-PNU | PEG4-na-EDA-PNU159682 | 2 | 45 | 47.5 | 35 | 108 | 50 | 380 |
| 2VhFc-PEG4-vc-PAB-DMAE-PNU | PEG4-vc-PAB-DMAE-PNU159682 | 2 | 11 | 3.13 | 4.5 | 40 | 4.7 | 32 |

The data in table 7 demonstrate that the PNU conjugates of the invention consistently have higher IC50 values than the prior art PEG4-vc-PAB-DMAE-PNU159682. The linker-payloads of the invention therefore give rise to more stable conjugates and/or less potent by-products and these protein drug conjugates should be less toxic to normal tissues.

### Example 3: Trastuzumab Conjugates

Trastuzumab mutants were produced in which a single serine residue in the Fc portion (position 442) is changed to a cysteine residue. This produces trastuzumab molecules with a unique thiol at position 442 of the heavy chain within the Fc portion for conjugation. Such mutants may be referred to as trastuzumab S442C or tras(S442C)

Two of the novel PNU payloads were conjugated to this molecule via the engineered cysteine using methods as described above to give the corresponding conjugates in good overall yield. Table 8 below outlines the properties of these conjugates.

TABLE 8
Analysis of tras(S442C)-PNU conjugates.

| | HC deglycosylated reduced mass, Da | | DAR by | Yield | HMW agg |
|---|---|---|---|---|---|
| Payload | Expected | Observed | MS | % | % |
| MA-PEG4-vc-PAB- | 50575 | 50578.1 | 1.02 | 27 | 8.2 |
| EDA-PNU159682 | | | | | |
| MA-PEG4-va-EDA- | 50339.7 | 50342.6 | 1.02 | 45 | 4.6 |
| PNU159682 | | | | | |

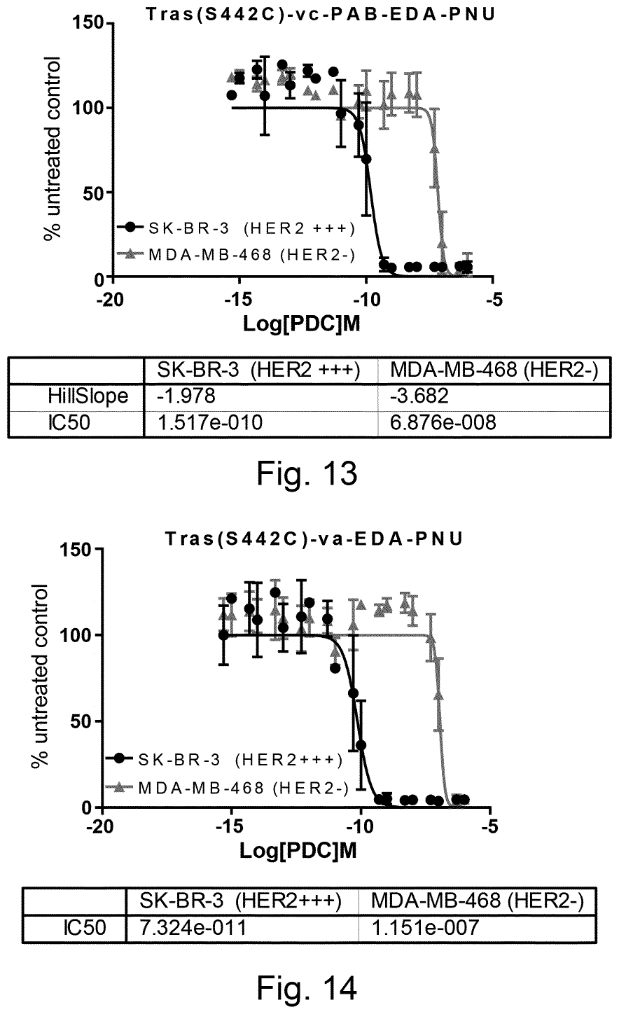

Furthermore, potency of the tras(S442C)-PNU conjugates in killing HER2 positive cells lines was investigated. FIGS. 13 and 14 show that both tras(S442C)-PNU conjugates selectively killed the HER2 positive cell line SK-BR-3 and had little effect on the HER2 negative cell line MDA-MB-468.

### Example 4: VNAR-PNU Conjugates

Multimeric and bi-paratopic VNAR constructs were generated with a C-terminal his myc tag containing an engineered cysteine for site specific labelling. Proteins were treated with 2 mM TCEP and purified by (MAC.

Binding kinetics for binding of the multimeric VNAR proteins to ROR1-hFc or ROR2-hFc fusion proteins (extracelluar domains) were determined using a Pioneer Surface Plasmon Resonance (SPR) instrument (SensiQ/Pall ForteBio) or the Biolayer Interferometry (BLI) Octet K2 system (ForteBio) as previously described.

TABLE 9
SPR data for binding of multimeric
VNARs to human ROR1 and human ROR2

| | hROR1 | | | |
|---|---|---|---|---|
| Molecule | Ka (M⁻¹s⁻¹) | Kd (s⁻¹) | KD (nM) | hROR2 |
| BA11-B1-D3 | 1.44E+05 | <1.0E−07 | <0.05 | No |
| (WbG4S) | | | | binding |
| P3A1-BA11-D3 | 1.28E+05 | 3.23E−04 | 2.53 | No |
| (WbG4SGM) | | | | binding |
| P3A1-BA11-D3 | 2.50+05 | 2.25−04 | 0.90 | No |
| ([G4S]5) | | | | binding |
| 2V-BA11-2V | No binding | No binding | No binding | No |
| (WbG4SGM) | | | | binding |

Conjugations were performed using 4 equivalents of PNU, room temperature for 1 hour and purified by SEC. Final conjugates analysed by analytical HIC, analytical SEC, and LC-MS.

TABLE 10
Characterisation of multimeric and bi-paratopic conjugates.

| | | | | | | Conjugate | HMW |
|---|---|---|---|---|---|---|---|
| | | Mass, Da | | DAR | | Yield | Aggregates |
| Protein | Payload | Expected | Observed | HIC | UV | % | % |
| BA11-B1-D3 | MA-PEG4-vc-PAB-EDA-PNU159682 | 43,026 | 43,046.2 | 1 | 0.60 | 23 | 4.8 |
| (WbG4S) | | | | | | | |
| | MA-PEG4-va-EDA-PNU159682 | 42,791 | 42,793.2 | 1 | 1.03 | 34 | 4.1 |
| P3A1-BA11-D3 | MA-PEG4-vc-PAB-EDA-PNU159682 | 41,821 | 41,821.1 | 0.88 | 0.61 | 34 | 2.1 |
| (WbG4SGM) | MA-PEG4-va-EDA-PNU159682 | 41,586 | 41,585.7 | 0.9 | 0.80 | 49 | 3.8 |
| P3A1-BA11-D3 | MA-PEG4-vc-PAB-EDA-PNU159682 | 42,982 | 42,980.6 | 1 | 0.66 | 22 | 15.1 |
| ([G4S]5) | MA-PEG4-va-EDA-PNU159682 | 42,747 | 42,744.1 | 1 | 0.60 | 32 | 9 |
| 2V-BA11-2V | MA-PEG4-vc-PAB-EDA-PNU159682 | 42,601 | 42,602.4 | 0.9-1 | 0.62 | 40 | 6.3 |
| (WbG4SGM) | MA-PEG4-va-EDA-PNU159682 | 42,366 | 42,367.2 | 0.89-1 | 0.73 | 45 | 6.6 |

The linkers between the VNAR domains are (G₄S)₅ [denoted by([G4S]5]; PGVQPSPGGGGS [denoted by (WbG4S)] (SEQ ID NO: 50); PGVQPAPGGGGS [denoted by (WbG4SGM)] (SEQ ID NO: 51).

Payloads were conjugated to the unique free thiol introduced at the C-terminal region of the protein through incorporation of a C-terminal his myc tag containing an engineered cysteine (sequence either QACKAHHHHHHGAEFEQKLISEEDL (SEQ ID NO: 52) or QACGAHHHHHHGAEFEQKLISEEDL (SEQ ID NO: 53)).

Binding of the multimeric conjugates to the surface of ROR1^{hi} A549 lung adenocarcinoma cells was assessed by flow cytometry. Adherent human cancer cells were detached from tissue culture flasks by incubating with 0.1% EDTA/PBS solution at 37° C. for ^{˜}10 minutes or until cells detached easily. Cells were re-suspended in 5 ml ice-cold PBS/2% FCS in 15 ml tubes and centrifuged at 1500 rpm for 5 mins at 4′C. Supernatant was removed and the cell pellet re-suspended in 1-2 ml of PBS/2% FCS. A cell count was performed using a Z1 Coulter Particle Counter (Beckman Coulter) and 5×10\hat{ }5 cells were aliquoted per test sample into a 96 well plate. Cells were incubated with 100 μl of the VNAR (His6Myc tagged) or corresponding VNAR conjugates at the indicated concentrations, plus controls for 1 hour on ice. The sample plate was centrifuged at 2000 rpm for 5 mins. The supernatant was removed and a wash performed by re-suspending the cell pellets in 0.25 mL of ice-cold PBS/2% FCS using a multichannel pipette. Samples were again centrifuged at 2000 rpm for 5 min at 4° C. Supernatant was removed and two further washes performed as described. After the final wash and centrifugation step, excess liquid was removed by blotting the plate on tissue paper. 100 μl of anti-×6His tag Ab (Abcam) was added per cell pellet as appropriate to bind to the VNAR (His6Myc tagged) or corresponding VNAR conjugates and incubated on ice for 30 mins. Wash steps were performed as described previously. PE-anti-mouse antibody (JIR) was used to detect binding of the VNAR (His6Myc tagged) and corresponding VNAR conjugates by incubating with the appropriate samples for 30 min on ice in the dark. Wash steps were performed as described previously. Cell pellets were re-suspended in 0.3 ml of ice-cold PBS/2% FCS and left on ice in the dark prior to analysis on a Merck-Millipore Guava EasyCyte HT flow cytometer.

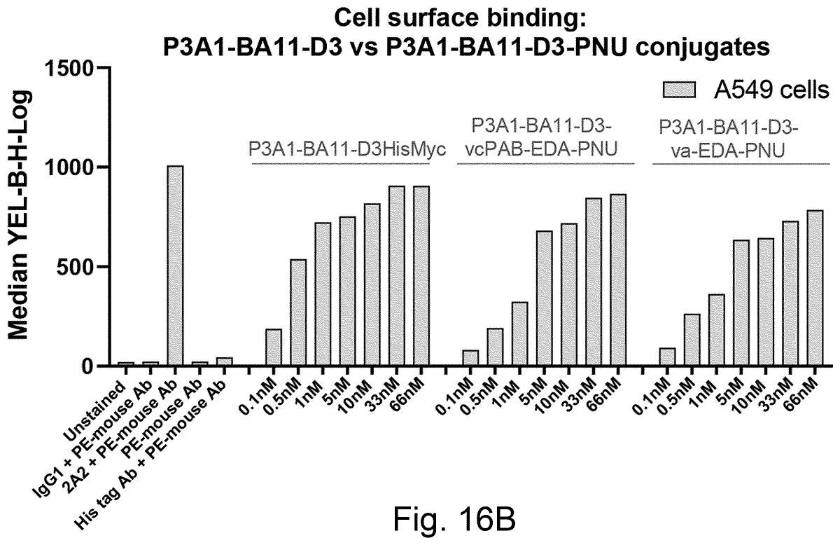

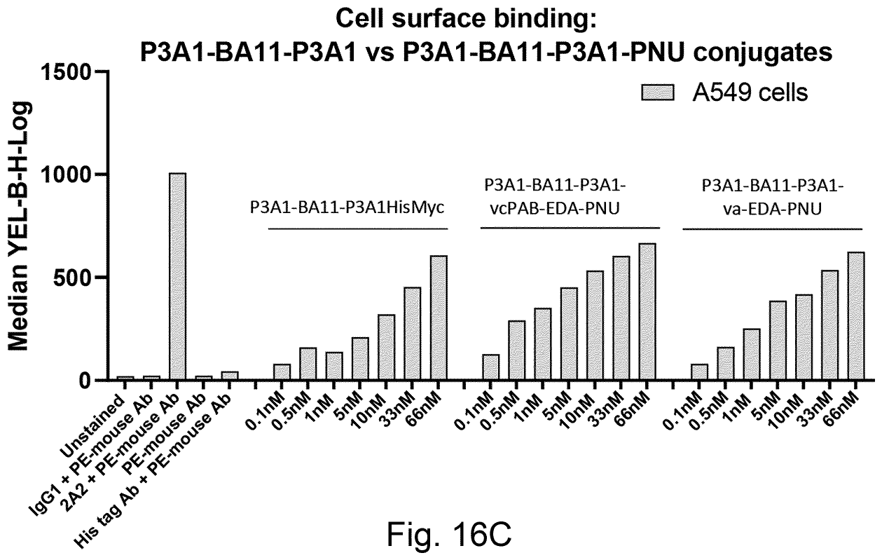

FIG. 16 shows that the VNAR multimers, conjugated with either vc-PAB-EDA-PNU or va-EDA-PNU maintain binding to ROR1 on the surface of the cancer cells.

### Example 5: Cathepsin B Treatment of Protein-PNU Conjugates

Cathepsin B treatment of VNAR-hFc-vc-PAB-EDA-PNU conjugates and the corresponding conjugates of the multimeric proteins liberated free EDA-PNU159682 derivative in a quantitative fashion. Whereas, VNAR-hFc-va-EDA-PNU conjugates and the corresponding conjugates of the multimeric proteins were completely stable to Cathepsin B treatment.

TABLE 11
Payload release after treatment with Cat B

| | Conc of | Theoretical | Average |
|---|---|---|---|
| | protein | conc of | Conc EDA |
| Conjugate | and DAR | payload (uM) | Released (uM) |
| B1hFc(S239C)-vc-PAB- | 10 uM | 21.0 | 21.3 |
| EDA-PNU | DAR 2.1 | | |
| B1hFc(S239C)-va-EDA- | 10 uM | 20.0 | <LLOQ |
| PNU | DAR 2.0 | | |
| 2VhFc(S239C)-vc_PAB- | 5 uM | 10.5 | 11.8 |
| EDA-PNU | DAR 2.1 | | |
| P3A1hFc(S239C)-vc-PAB- | 10 uM | 22.0 | 20.1 |
| EDA-PNU | DAR 2.2 | | |
| P3A1hFc(S239C)-va-EDA- | 10 uM | 21.0 | 0.36 (1.7%) |
| PNU | DAR 2.1 | | |

Only the vc-PAB-EDA-PNU releases payload on treatment with CatB as expected for the conditions used in this in vitro assay. The release is quantitative—as the conjugates have a drug to antibody ratio of 2, then twice the concentration of payload released versus the concentration of conjugate is expected after CatB treatment. This expectation matches the data shown in Table 11 for vc-PAB-EDA-PNU conjugates.

### Example 6: Plasma Stability of Protein-PNU Conjugates

Different P3A1-hFc-PNU conjugates were incubated at 37° C. in mouse plasma at a final concentration of 4 μM of protein. Samples were analysed by LC-MS as a function of time and the amount of released PNU159682 and EDA-PNU159682 derivative was quantified by reference to calibration standards.

TABLE 12
Stability of protein-PNU conjugates after incubation in mouse plasma

| | Concentration (nM) of free PNU payload detected by LC-MS | | | | | |
|---|---|---|---|---|---|---|
| Conjugate | 4 h | 24 h | 48 h | 72 h | 96 h | 120 h |
| P3A1hFc-vc-PAB-DMAE-PNU159682 | 13 | 68 | 121 | 145 | 170 | 217 |
| P3A1-hFc-vc-PAB-EDA-PNU159682 | 21 | 84 | 108 | 107 | 101 | 114 |
| P3A1-hFc-va-EDA-PNU159682 | 3 | 4 | 1 | 2 | 5 | 3 |

As shown in Table 12, conjugates with the va-EDA-PNU159682 payload showed excellent mouse plasma stability with little to none PNU derivatives released with time. For both the vc-PAB-DMAE-PNU and vc-PAB-EDA-PNU conjugates liberation of some free payload could be detected as a function of time, with 217 nM PNU159682 and 114 nM of EDA-PNU159682 derivative detected after 120 h respectively. Note, in a parallel study, we calculated the mouse plasma half-lives of the free PNU159682 and EDA-PNU159682 derivatives to be 33 h and 116 h respectively. Indicating that the absolute amount of free PNU payload released for the vc-PAB-DMAE-PNU conjugate is underestimated with respect to the vc-PAB-EDA-PNU conjugate.

Different conjugates were incubated at 37° C. for 168 h in human plasma at a final concentration of 4 μM or 2 μM of protein. Samples were analysed by LC-MS as a function of time up to and including 168 h and the amount of released PNU159682 and EDA-PNU159682 derivative was quantified by reference to calibration standards.

TABLE 13
Stability of protein-PNU conjugates after incubation in human plasma

| | | | Maximum | |
|---|---|---|---|---|
| | | | theoretical | |
| | Concentration | PDC | payload release | |
| | of PDC(uM) | DAR | (uM) | Observed EDA payload release |
| P3A1-hFc(S239c)-vc- | 4 | 2.2 | 8.8 | EDA-PNU not detected above LLOQ |
| PAB-EDA-PNU | | | | LLOQ = 0.156 uM (1.8% of maximal |
| | | | | payload release) |
| P3A1-hFc(S239c)-va- | 4 | 2.1 | 8.4 | no EDA-PNU detected at any time points |
| EDA-PNU | | | | LLOQ = 0.156 uM (1.9% of maximal |
| | | | | payload release) |
| B1h-Fc(S239C)- | 4 | 2.1 | 8.4 | EDA-PNU not detected above LLOQ |
| vcPAB-EDA-PNU | | | | LLOQ = 0.1 uM (1.2% of maximal |
| | | | | payload release) |
| B1-hFc(S239C)-va- | 4 | 2.0 | 8.0 | EDA-PNU not detected above LLOQ |
| EDA-PNU | | | | LLOQ = 0.156 uM (1.95% of maximal |
| | | | | payload release) |
| P3A1-hFc(442)-va- | 2 | 1.4 | 2.8 | No EDA-PNU detected at any time points |
| EDA-PNU | | | | LLOQ = 0.1 uM (3.57% of maximal |
| | | | | payload release) |

As shown in Table 13, conjugates showed excellent human plasma stability with no detectable amounts of PNU derivatives released over the timecourse of the experiment. The half-life of EDA-PNU in human plasma is 172.25h (average of 4 different experiments).

### Example 7: In Viva Efficacy of Protein-Drug Conjugates in a Patient-Derived Xenograft Model of Pleural Mesothelioma

An efficacy study in the ROR1+PXF-1118 patient-derived pleural mesothelioma xenograft model was performed by Charles River Laboratories (Freiburg).

Tumour fragments obtained from xenografts in serial passage in nude mice were implanted subcutaneously into female NMRI nu/nu mice (CrI:NMRI-Foxn1^{nu}). Mice were monitored until the tumour implants reached the study volume recruitment criteria of 50-250 mm³, preferably 150-200 mm³ in a sufficient number of animals. Mice were randomised to treatment groups such that there was no statistical difference between tumour volumes in each group. Randomisation was designated as Day 0 of the experiment. Mice were treated with vehicle or with the protein-drug conjugates B1-hFc-vc-PAB-EDA-PNU or B1-hFc-va-EDA-PNU at 0.3 mg/kg by i.v. injection on days 1, 4, 7, 10, 18. All mice received single-dose priming with mouse IgG, administered intravenously (i.v.) at 29 mg/kg 20 hours ahead of the first PDC dose.

The absolute tumour volumes (ATVs) were determined by two-dimensional measurement with a digital caliper on the day of randomisation and then three times a week. Tumour volumes were calculated according to the formula:

Tumour volume=(L×W²)×0.5

where L=largest diameter and W=width (perpendicular diameter) of the tumour (in mm).

Animals were routinely weighed three times a week, and on days when doses were administered. Mice were observed and documented daily for changes in physical appearance, behaviour and adverse clinical signs and general welfare in line with local and best veterinary practice guidelines.

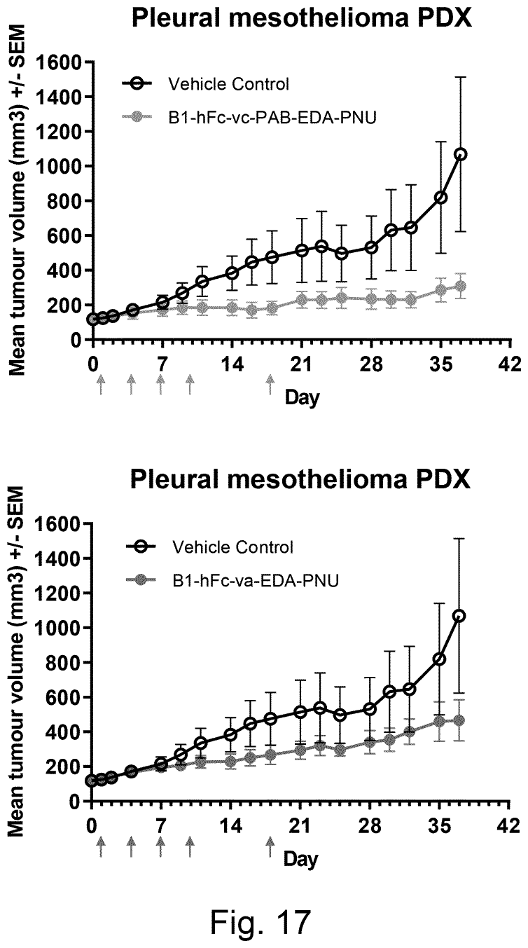

FIG. 17 shows the effect of the protein-drug conjugates on tumour growth versus vehicle control. Both B1-hFc-vc-PAB-EDA-PNU and B1-hFc-va-EDA-PNU are well tolerated and significantly inhibited the growth of tumours in this ROR1+pleural mesothelioma PDX model. PDC molecules targeting ROR1+ve pleural mesothelioma patient-derived tumours display good anti-tumour efficacy.

### Example 8: In Vivo Efficacy of Protein-Drug Conjugates in Patient-Derived Xenograft Model of Triple Negative Breast Cancer (TNBC)

An efficacy study in the ROR1+HBCx-28 patient-derived TNBC xenograft model was performed by XenTech (Paris).

Outbred athymic (nu/nu) female mice (HSD: Athymic Nude-Foxn1^{nu}) were implanted subcutaneously with tumours of the same in vivo passage. Mice were monitored until the tumour implants reached the study volume recruitment criteria of 60-200 mm³, preferably 75-196 mm³ in a sufficient number of animals. Mice were randomised to treatment groups such that there was no statistical difference between tumour volumes in each group. Randomisation was designated as Day 0 of the experiment. Mice were treated with vehicle or with the protein-drug conjugates B1-hFc-vc-PAB-EDA-PNU or B1-hFc-va-EDA-PNU at 0.3 mg/kg by i.v. injection on days 2, 5, 8, 12, 15 with all mice pre-primed with mouse IgG 20h before first PDC dose. Tumour volume was evaluated by measuring perpendicular tumour diameters, with a caliper, three times a week during the experimental period until D55, then measured and weighed twice a week until the end of the experiment (i.e. Day 103). Absolute tumour volume (ATV) was calculated using the formula TV (mm³)=[length (mm)×width (mm)²]×0.5, where the length and the width are the longest and the shortest perpendicular diameters of the tumour measured perpendicularly, respectively. All animals were weighed at the same time as tumour size measurement. Mice were observed and documented daily for changes in physical appearance, behaviour, adverse clinical signs and general welfare in line with local welfare and best veterinary practice guidelines.

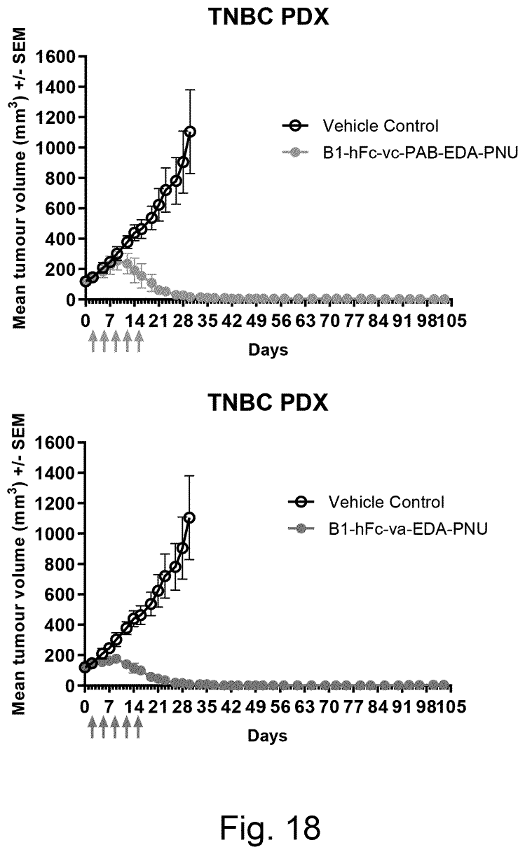

FIG. 18 shows the effect of the protein-drug conjugates on tumour growth versus vehicle control. Both B1-hFc-vc-PAB-EDA-PNU and B1-hFc-va-EDA-PNU are well tolerated and show highly statistically significant in vivo efficacy in this ROR1+TNBC PDX model In addition, complete and durable regressions were observed for both agents including tumour ‘cures’ that were sustained for the full length of the study (103 days). PDC molecules targeting ROR1+ve TNBC patient-derived tumours display robust anti-tumour efficacy with complete and durable tumour regressions (including ‘cures’).

1. An anthracycline (PNU) derivative of formula (I): 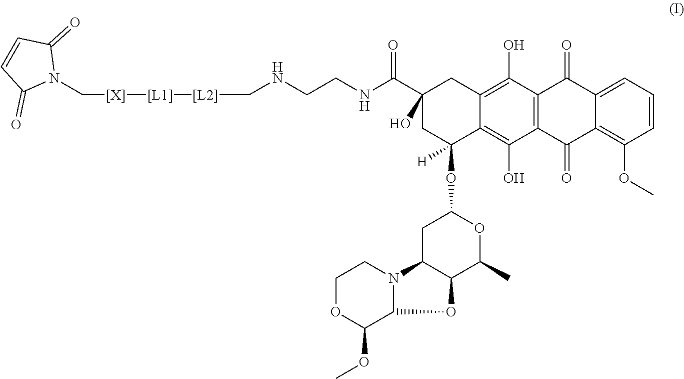
wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, any amino acid except glycine, and combinations thereof, wherein the anthracycline (PNU) derivative of formula (I) comprises [L1], [L2] or [L1] and [L2].

2. (canceled)

3. The anthracycline derivative of claim 1, wherein [X] is selected from the group comprising polyethylene glycol, 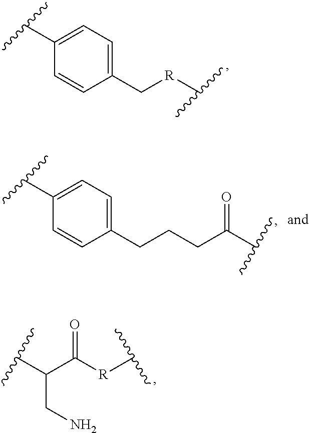
wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

4. The anthracycline derivative of claim 1, wherein [X] is polyethylene glycol.

5. The anthracycline derivative of claim 1, wherein [L2] is p-aminobenzyloxycarbonyl (PAB) or alanine.

6. The anthracycline derivative of claim 1, wherein the PNU derivative has a structure selected from:

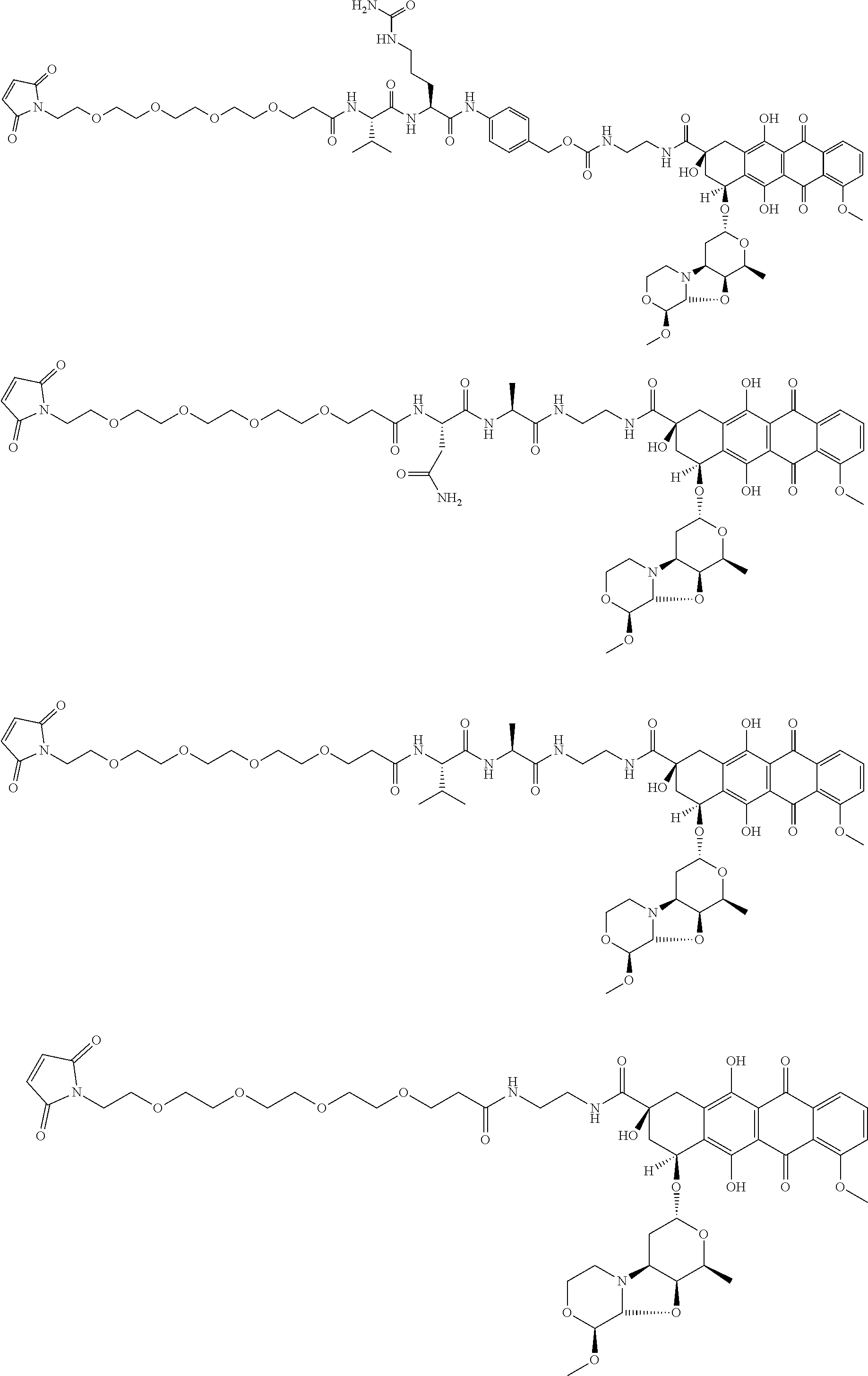

7. An anthracycline (PNU) derivative of formula (IV): 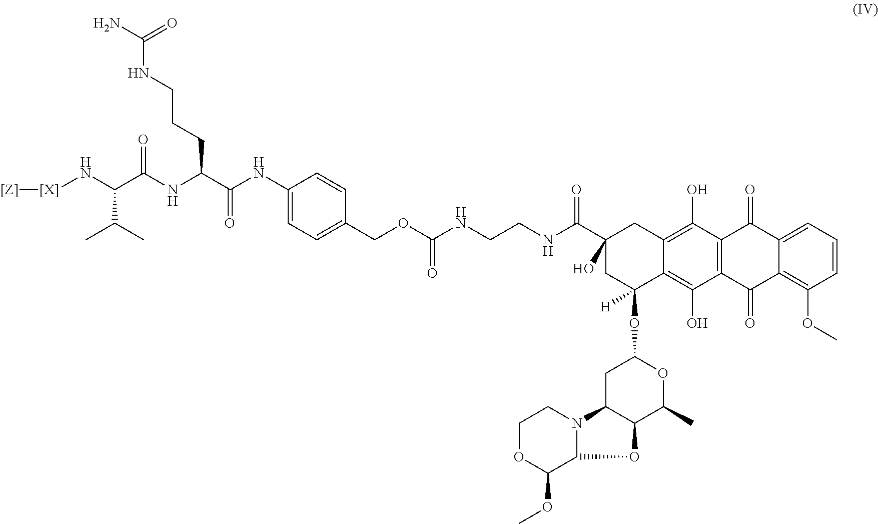
wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
and wherein [Z] is a reactive group suitable for use in a conjugation reaction.

8. The anthracycline derivative of claim 7, wherein [Z] is selected from the group consisting of a maleimide, an alkyl halide, a sulphydryl group, an activated disulphide, an amino group, an alkyne group, an azido group, an aminoxy group, an aldehyde group and a ketone group.

9. The anthracycline derivative of claim 7, wherein [Z] is selected from the group consisting of polyGly and a primary amine.

10. The anthracycline derivative of claim 7, wherein [X] is selected from the group comprising polyethylene glycol, 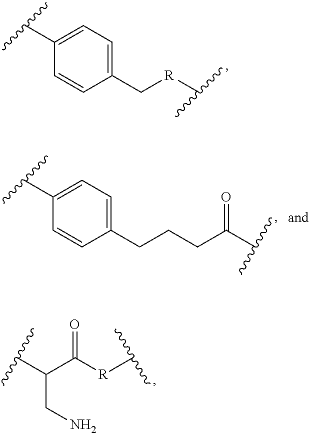
wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

11. The anthracycline derivative of claim 7, wherein [X] is polyethylene glycol.

12. A target-binding molecule-drug conjugate, comprising a specific antigen binding protein and an anthracycline (PNU) derivative, wherein the target-binding molecule-drug conjugate has the structure of formula (II): 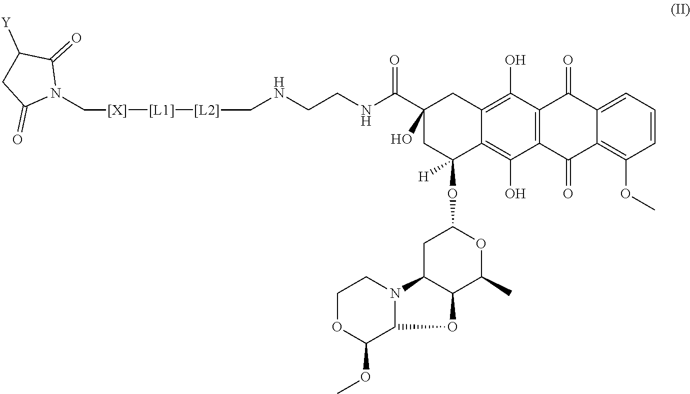
wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, any amino acid except glycine, and combinations thereof; and
Y is a target-binding molecule, wherein the target-binding molecule-drug conjugate of formula (II) comprises [L1], [L2] or [L1] and [L2].

13. (canceled)

14. The target-binding molecule-drug conjugate of claim 12, wherein [X] is selected from the group comprising polyethylene glycol, 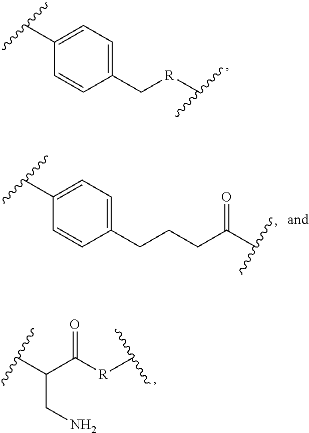
wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

15. The target-binding molecule-drug conjugate of claim 12, wherein [X] is polyethylene glycol.

16. The target-binding molecule-drug conjugate of claim 12, wherein [L2] is p-aminobenzyloxycarbonyl (PAB) or alanine.

17. The target-binding molecule-drug conjugate of claim 12, wherein the PNU derivative has a structure selected from:

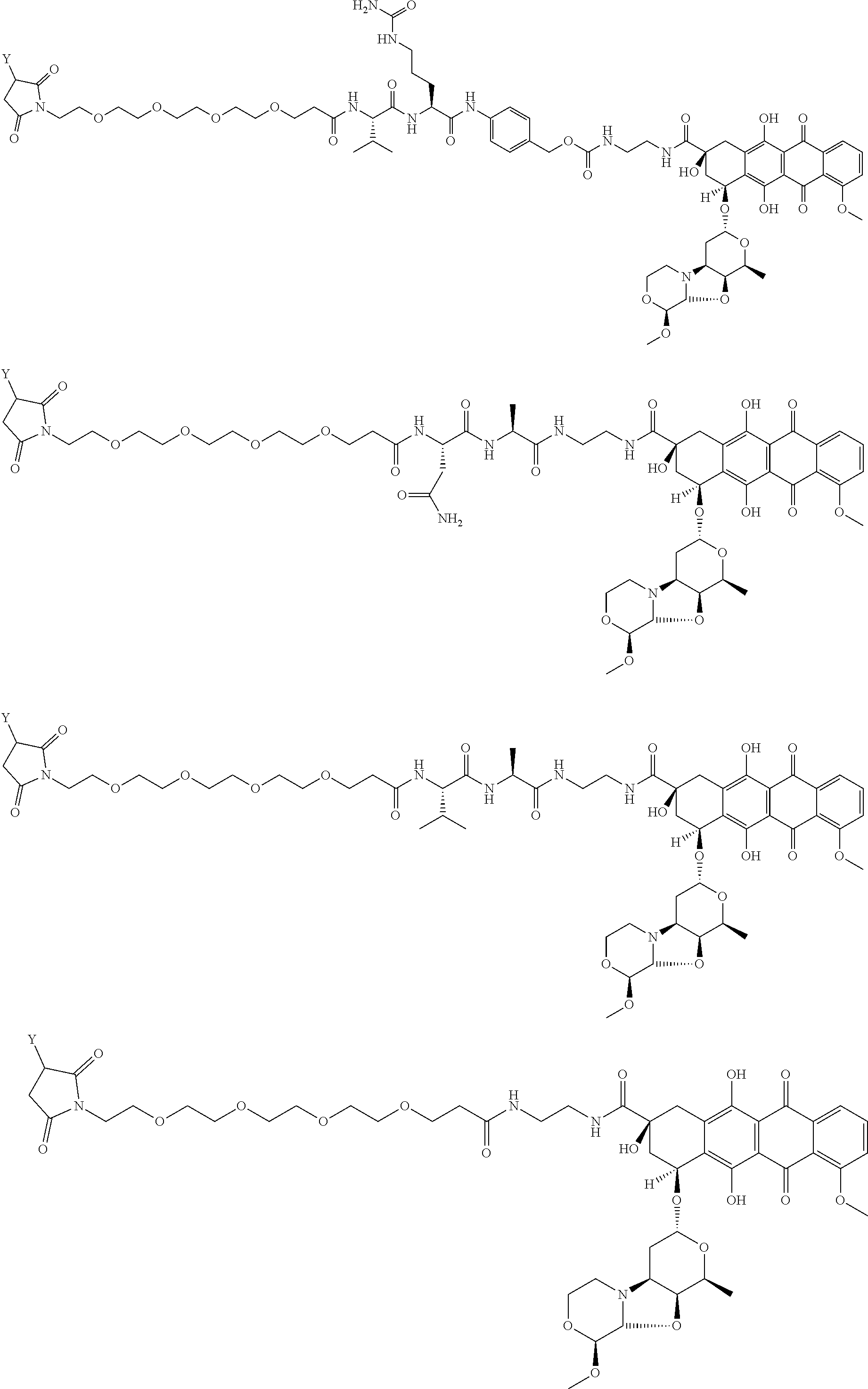

18. A target-binding molecule-drug conjugate, comprising a specific antigen binding protein and an anthracycline (PNU) derivative, wherein the target-binding molecule-drug conjugate has the structure of formula (V): 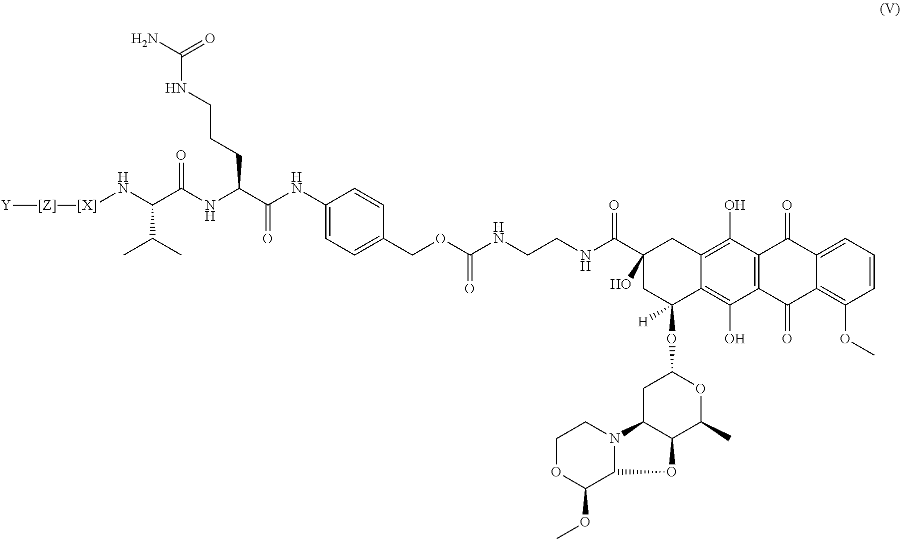
wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[Z] is a linker derived from a reactive group used to conjugate the anthracycline (PNU) derivative and the target-binding molecule; and
Y is a target binding molecule.

19. The anthracycline derivative of claim 18, wherein [Z] is selected from the group consisting of a disulphide bond, an amide bond, an oxime bond, a hydrazone bond, a thioether bond, a 1, 2,3 triazole and polyGly.

20. The target-binding molecule-drug conjugate of claim 18, wherein [X] is selected from the group comprising polyethylene glycol, 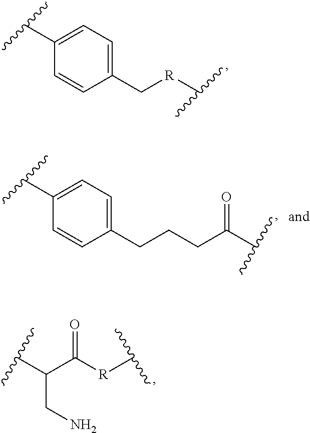
wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

21. The target-binding molecule-drug conjugate of claim 18, wherein [X] is polyethylene glycol.

22. The target-binding molecule-drug conjugate of claim 12, wherein the target-binding molecule is a protein and anthracycline (PNU) derivative is conjugated to a thiol-containing amino acid residue in the amino acid sequence of the protein or wherein the PNU derivative is conjugated via a thiol moiety incorporated by chemical modification at the N-terminus or C-terminus of the amino acid sequence of the protein.

23. The target-binding molecule-drug conjugate according to claim 12, wherein the target-binding molecule is a binding protein selected from the group comprising an immunoglobulin or antibody, an immunoglobulin Fc region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager (BiTE), an intein, a VNAR domain, a single domain antibody (sdAb), a VH domain, or a scaffold protein.

24. The target-binding molecule-drug conjugate according to claim 12, where the target-binding molecule binds to receptor tyrosine kinase-like orphan receptor 1 (ROR1).

25. The target-binding molecule-drug conjugate of claim 12, wherein the target-binding molecule is a specific antigen binding protein comprising an amino acid sequence represented by the formula (III):
FW1-CDR1-FW2-HV2-FW3a-HV4-FW3b-CDR3-FW4 (III)
wherein
FW1 is a framework region
CDR1 is a CDR sequence
FW2 is a framework region
HV2 is a hypervariable sequence
FW3a is a framework region
HV4 is a hypervariable sequence
FW3b is a framework region
CDR3 is a CDR sequence
FW4 is a framework region.

26. The target-binding molecule-drug conjugate according to claim 25, wherein the specific antigen binding protein binds receptor tyrosine kinase-like orphan receptor 1 (ROR1), does not bind to receptor tyrosine kinase-like orphan receptor 2 (ROR2), binds to both human ROR1 and murine ROR1 (mROR1), or binds to deglycosylated ROR1.

27-29. (canceled)

30. The target-binding molecule-drug conjugate according to claim 26, wherein the ROR1-specific antigen binding protein does not bind to a linear peptide sequence selected from:
(SEQ ID NO: 34)
YMESLHMQGEIENQI
(SEQ ID NO: 35)
CQPWNSQYPHTHTFTALRFP
(SEQ ID NO: 36)
RSTIYGSRLRIRNLDTTDTGYFQ
(SEQ ID NO: 37)
QCVATNGKEVVSSTGVLFVKFGPPPTASPGYSDEYE

31. The target-binding molecule-drug conjugate according to claim 26, wherein
FW1 is a framework region of from 20 to 28 amino acids
CDR1 is a CDR sequence selected from DTSYGLYS (SEQ ID NO: 1), GAKYGLAA (SEQ ID NO: 2), GAKYGLFA (SEQ ID NO: 3), GANYGLAA (SEQ ID NO: 4), or GANYGLAS (SEQ ID NO: 5)
FW2 is a framework region of from 6 to 14 amino acids
HV2 is a hypervariable sequence selected from TTDWERMSIG (SEQ ID NO: 6), SSNQERISIS (SEQ ID NO: 7), or SSNKEQISIS (SEQ ID NO: 8)
FW3a is a framework region of from 6 to 10 amino acids
HV4 is a hypervariable sequence selected from NKRAK (SEQ ID NO: 9),
NKRTM (SEQ ID NO: 10), NKGAK (SEQ ID NO: 11), or NKGTK (SEQ ID NO: 12)
FW3b is a framework region of from 17 to 24 amino acids
CDR3 is a CDR sequence selected from QSGMAISTGSGHGYNWY (SEQ ID NO: 13), QSGMAIDIGSGHGYNWY (SEQ ID NO: 14), YPWAMWGQWY (SEQ ID NO: 15), VFMPQHWHPAAHWY (SEQ ID NO: 16), REARHPWLRQWY (SEQ ID NO: 17), or YPWGAGAPWLVQWY (SEQ ID NO: 18)
FW4 is a framework region of from 7 to 14 amino acids or a functional variant thereof with at least 45% sequence identity thereto,

32. The target-binding molecule-drug conjugate according to claim 26, wherein FW1 is selected from:
ASVNQTPRTATKETGESLTINCVLT (SEQ ID NO: 19),
AKVDQTPRTATKETGESLTINCVLT (SEQ ID NO: 20),
TRVDQTPRTATKETGESLTINCVVT (SEQ ID NO: 21),
TRVDQTPRTATKETGESLTINCVLT (SEQ ID NO: 22),
ASVNQTPRTATKETGESLTINCVVT (SEQ ID NO: 23),
TRVDQSPSSLSASVGDRVTITCVLT (SEQ ID NO: 24) or
ASVTQSPRSASKETGESLTITCRVT (SEQ ID NO: 56), FW2 is selected from:
TSWFRKNPG (SEQ ID NO: 25), or TYWYRKNPG (SEQ ID NO: 26); FW3a is selected from: GRYVESV (SEQ ID NO: 27), or GRYSESV (SEQ ID NO: 28), FW3b is selected from: SFSLRIKDLTVADSATYYCKA (SEQ ID NO: 29), SFTLTISSLQPEDSATYYCRA (SEQ ID NO: 30), SFTLTISSLQPEDFATYYCKA (SEQ ID NO: 31) or SFSLRISSLTVEDSATYYCKA (SEQ ID NO: 57), and FW4 is selected from:
DGAGTVLTVN (SEQ ID NO: 32), DGAGTKVEIK (SEQ ID NO: 33) or DGQGTKLEVK (SEQ ID NO: 58); or functional variants thereof with a sequence identity of at least 45%.

33. The target-binding molecule-drug conjugate according to claim 26, wherein the ROR1-specific antigen binding molecule comprises an amino acid sequence selected from:
(SEQ ID NO: 39)
ASVNQTPRTATKETGESLTINCVLTDTSYGLYSTSWFRKNPGTTDWERM
SIGGRYVESVNKRAKSFSLRIKDLTVADSATYYCKAQSGMAISTGSGHG
YNWYDGAGTVLTVN;
(SEQ ID NO: 40)
AKVDQTPRTATKETGESLTINCVLTDTSYGLYSTSWFRKNPGTTDWERM
SIGGRYVESVNKRAKSFSLRIKDLTVADSATYYCKAQSGMAIDIGSGHG
YNWYDGAGTVLTVN;
(SEQ ID NO: 41)
TRVDQTPRTATKETGESLTINCVVTGAKYGLAATYWYRKNPGSSNQERI
SISGRYVESVNKRTMSFSLRIKDLTVADSATYYCKAYPWAMWGQWYDGA
GTVLTVN;
(SEQ ID NO: 42)
TRVDQTPRTATKETGESLTINCVVTGAKYGLFATYWYRKNPGSSNQERI
SISGRYVESVNKRTMSFSLRIKDLTVADSATYYCKAVFMPQHWHPAAHW
YDGAGTVLTVN;
(SEQ ID NO: 43)
TRVDQTPRTATKETGESLTINCVLTDTSYGLYSTSWFRKNPGTTDWERM
SIGGRYVESVNKGAKSFSLRIKDLTVADSATYYCKAREARHPWLRQWYD
GAGTVLTVN;
(SEQ ID NO: 44)
ASVNQTPRTATKETGESLTINCVVTGANYGLAATYWYRKNPGSSNQERI
SISGRYVESVNKRTMSFSLRIKDLTVADSATYYCKAYPWGAGAPWLVQW
YDGAGTVLTVN;,
(SEQ ID NO: 45)
TRVDQSPSSLSASVGDRVTITCVLTGANYGLASTYWYRKNPGSSNKEQI
SISGRYSESVNKGTKSFTLTISSLQPEDSATYYCRAYPWGAGAPWLVQW
YDGAGTKVEIK;
(SEQ ID NO: 46)
TRVDQSPSSLSASVGDRVTITCVLTGANYGLASTYWYRKNPGSSNQERI
SISGRYSESVNKRTMSFTLTISSLQPEDSATYYCRAYPWGAGAPWLVQW
YDGAGTKVEIK;
(SEQ ID NO: 47)
TRVDQSPSSLSASVGDRVTITCVLTDTSYGLYSTSWFRKNPGTTDWERM
SIGGRYVESVNKGAKSFTLTISSLQPEDFATYYCKAREARHPWLRQWYD
GAGTKVEIK;
(SEQ ID NO: 48)
TRVDQSPSSLSASVGDRVTITCVLTDTSYGLYSTYWYRKNPGSSNKEQI
SISGRYSESVNKGTKSFTLTISSLQPEDSATYYCRAREARHPWLRQWYD
GAGTKVEIK;
(SEQ ID NO: 49)
TRVDQSPSSLSASVGDRVTITCVLTDTSYGLYSTYWYRKNPGTTDWERM
SIGGRYSESVNKGAKSFTLTISSLQPEDSATYYCRAREARHPWLRQWYD
GAGTKVEIK;
(SEQ ID NO: 59)
ASVTQSPRSASKETGESLTITCRVTGANYGLAATYWYRKNPGSSNQERI
SISGRYSESVNKRTMSFSLRISSLTVEDSATYYCKAYPWGAGAPWLVQW
YDGQGTKLEVK;
or a functional variant thereof with a sequence identity of at least 45%.

34. The target-binding molecule-drug conjugate of claim 26, wherein the ROR1-specific antigen binding protein is humanized or de-immunized.

35-38. (canceled)

39. A method of treatment of a disease in a patient in need of treatment comprising administration to said patient of a therapeutically effective dosage of a target-binding molecule-drug conjugate according to claim 24.

40. The method of claim 39, wherein the disease is cancer.

41. The target-binding molecule-drug conjugate according to claim 12, wherein the target-binding molecule is an antibody, an antibody that binds HER-2, trastuzumab or a derivative thereof.

42-43. (canceled)

44. A pharmaceutical composition comprising a target-binding molecule-drug conjugate according to claim 24, and at least one other pharmaceutically acceptable ingredient.
